(19) United States
(12) Patent Application Publication
(10) Pub. No.: US 2023/0233738 A1
(43) Pub. Date: Jul. 27, 2023

(54) COMPOSITIONS AND METHODS FOR AUGMENTING AUTOLOGOUS FAT GRAFTS

(71) Applicant: Duke University, Durham, NC (US)

(72) Inventors: Stefan Roberts, Raleigh, NC (US); Ashutosh Cilkoti, Durham, NC (US); Bruce Klitzman, Littleton, NC (US); Scott Hollenbeck, Durham, NC (US)

(21) Appl. No.: 18/007,879

(22) PCT Filed: Jun. 4, 2021
(86) PCT No.: PCT/US2021/035823

§ 371 Date: Dec. 2, 2022

Related U.S. Application Data
(60) Provisional application No. 63/035,173, filed on Jun. 5, 2020.

(51) Int. Cl. A61L 27/36; A61L 27/22; C07K 14/00

(52) U.S. Cl. CPC A61L 27/3604; A61L 27/227; C07K 14/00; A61L 2300/252; A61L 2300/22

(57) ABSTRACT

Described herein are compositions and method for autologous adipose tissue grafting. In one embodiment, the composition comprises a recombinant partially ordered polypeptide (Fractomer) or “Fractomer” and adipose tissue from a subject. In one aspect, the Fractomer has the general structure of [(GXGVP)ₙ-α-helix]ₘ, where X can be any amino acid except proline and α-helix is any polyalanine based α-helix having about 5 to 50 Alanine residues. In another aspect, the Fractomer has the structure [(GXGVP)ₙ-GX¹(A)₂₅X¹]ₘ; where X is A or V; X¹ is K or D; n is an integer from 10 to 20; and m is an integer from 4 to 8.

### CROSS-REFERENCE TO RELATED APPLICATIONS

This application claims priority to U.S. Provisional Patent Application No. 63/035,173 filed on Jun. 5, 2020, which is incorporated by reference herein in its entirety.

### FEDERALLY SPONSORED RESEARCH

This invention was made with government support under grant number 1R41CA244110-01A1 awarded by the National Institutes of Health. The government has certain rights in the invention.

### REFERENCE TO SEQUENCE LISTING

This application is filed with a Computer Readable Form of a Sequence Listing in accord with 37 C.F.R. § 1.821(c). The text file submitted by EFS, “028193-9366-WO01_sequence_listing_2 Jun. 2021_ST25.txt,” was created on Jun. 2, 2021, contains 23 sequences, has a file size of 37.4 Kbytes, and is hereby incorporated by reference in its entirety.

### TECHNICAL FIELD

Described herein are compositions and method for autologous adipose tissue grafting. In one embodiment, the composition comprises a recombinant partially ordered polypeptide (Fractomer) or “Fractomer” and adipose tissue from a subject. In one aspect, the Fractomer has the general structure of [(GXGVP)ₙ-α-helix]ₘ, where X can be any amino acid except proline and α-helix is any polyalanine based α-helix having about 5 to 50 Alanine residues. In another aspect, the Fractomer has the structure [(GXGVP)ₙ-GX¹(A)₂₅X¹]ₘ; where X is A or V; X¹ is K or D; n is an integer from 10 to 20; and m is an integer from 4 to 8.

### BACKGROUND

Autologous fat grafting is a valuable option to treat contour irregularities and volume deficits in the nearly 6 million Americans undergoing reconstructive plastic surgery each year. Though preferred for their permanence and innate biocompatibility, fat grafts-especially large volume grafts such as those used in post-mastectomy patients-routinely require multiple surgeries due to the insufficient available volume of harvested tissue. Hence, there is a need for novel innovations relating to tissue grafts.

### SUMMARY

One embodiment described herein is a tissue matrix composition comprising: a recombinant partially ordered polypeptide (Fractomer); and adipose tissue. In one aspect, the Fractomer comprises: a plurality of disordered domains; and a plurality of structured domains. In another aspect, the disordered domain comprises a plurality of an amino acid sequence of (GXGVP)ₙ (SEQ ID NO:1), wherein X is any amino acid except proline and n is an integer greater than or equal to 1; and the structured domain comprises a polyalanine domain. In another aspect, the disordered domain comprises a plurality of an amino acid sequence of (GXGVP)ₙ (SEQ ID NO: 2), wherein X is Val (SEQ ID NO: 3), or Ala (SEQ ID NO: 4), or mixture of Ala and Val, and wherein n is an integer from 1 to 50. In another aspect, X is an alternating iteration of Ala and Val in a ratio from 10:1 to 1:10 (Ala:Val). In another aspect, X is an alternating iteration of Ala and Val in a ratio of 1:1 (SEQ ID NO: 5) or 1:4 (SEQ ID NO: 6) In another aspect, the polyalanine domain comprises (Ala)ₘ, wherein m is an integer from 5 to 50. In another aspect, the polyalanine domain comprises one or more of: (A)₂₅ (SEQ ID NO: 7); K(A)₂₅K (SEQ ID NO: 8); D(A)₂₅K (SEQ ID NO: 9); GD(A₂₅)K (SEQ ID NO: 10); or GK(A₂₅)K (SEQ ID NO: 11). In another aspect, the polypeptide comprises: [(GXGVP)ₙ-GX¹(A)₂₅X¹]ₘ; where X is A or V; X¹ is K or D; n is an integer from 10 to 20; and m is an integer from 4 to 8 ([(SEQ ID NO: 2)ₙ-(SEQ ID NO: 10 or 11)]ₘ). In another aspect, the polypeptide comprises one or more of: M[(GVGVP)₁₅-GD(A25)K]₆-GWP (SEQ ID NO: 12); M[(GVGVP)₁₅-GD(A25)K]₄-GWP (SEQ ID NO: 13); M[(GVGVP)₁₅-GK(A25)K]₆-GWP (SEQ ID NO: 14); M[(GVGVP)₁₅-GK(A25)K]₄-GWP (SEQ ID NO: 15); M[(G[A1:V1]GVP)₁₆-GD(A₂₅)K]₆-GWP (SEQ ID NO: 16); M[(G[A1:V1]GVP)₁₅-GD(A₂₅)K]₄-GWP (SEQ ID NO: 17); M[(G[V4:A1]GVP)₁₅-GD(A₂₅)K]s-GWP (SEQ ID NO: 18); or M[(G[V4:A1]GVP)₁₅-GD(A₂₅)K]₄-GWP (SEQ ID NO: 19). In another aspect, the polypeptide comprises one or more of: M[(GVGVP)₁₅-GD(A₂₅)K]₆-GWP (SEQ ID NO: 12); or M[(G[V4:A1]GVP)₁₅-GD(A₂₅)K]s-GWP (SEQ ID NO: 18). In another aspect, the Fractomer has a transition temperature of heating (T_{t-heating}) and a transition temperature of cooling (T_{t-cooling}). In another aspect, the transition temperature of cooling (T_{t-cooling}) is concentration-independent. In another aspect, the transition temperature of heating (T_{t-heating}) and the transition temperature of cooling (T_{t-cooling}) range from about 10° C. to about 45° C. In another aspect, the Fractomer forms a solid aggregate above the T_{t-heating}. In another aspect, the solid aggregate resolubilizes when cooled to below the T_{t-cooling}. In another aspect, the solid aggregate is a stable three-dimensional matrix. T In another aspect, the solid aggregate comprises a plurality of micropores. In another aspect, the plurality of micropores range in size from about 1 μm to about 150 μm. The composition of clause 1, wherein the composition comprises between about 200 μM and about 2 mM of Fractomer. In another aspect, the adipose tissue comprises lipoaspirate. In another aspect, the composition comprises a range of lipoaspirate from about 10% to about 90% by volume. In another aspect, the composition comprises a range of lipoaspirate from about 25% to about 75% by volume. In another aspect, the composition comprises about 50% by volume lipoaspirate. In another aspect, the composition comprises a mixture of Fractomer and lipoaspirate in a ratio ranging from about 1:9 to about 9:1. In another aspect, the composition comprises a mixture of Fractomer and lipoaspirate in a ratio ranging from about 1:3 to about 3:1. In another aspect, the composition comprises a mixture of Fractomer and lipoaspirate in a ratio of about 1:1. In another aspect, the composition is a shapeable liquid or semisolid. In another aspect, the composition is injectable or implantable. In another aspect, the composition is shapeable or moldable into 2- or 3-dimensional shapes, areas, or volumes. In another aspect, the Fractomer permits cell infiltration and vascularization of the adipose tissue.

Another embodiment described herein is a method of augmenting autologous fat grafts in a subject, the method comprising: administering to the subject a therapeutically effective amount of a composition comprising: a recombinant partially ordered polypeptide (Fractomer); and adipose tissue as described herein, such that the autologous fat grafts are augmented in the subject.

Another embodiment described herein is a method of augmenting an autologous fat graft in a subject, the method comprising: co-administering to the subject a therapeutically effective amount of a recombinant partially ordered polypeptide (Fractomer) and a therapeutically effective amount of adipose tissue. In one aspect, the adipose tissue comprises a lipoaspirate. In another aspect, the Fractomer and adipose tissue are administered concurrently or sequentially. In another aspect, the Fractomer and adipose tissue are administered sequentially, and the Fractomer is administered prior to the administration of the adipose tissue. In another aspect, the Fractomer and adipose tissue are administered sequentially, and the adipose tissue is administered prior to the administration of the Fractomer. In another aspect, the Fractomer and adipose tissue are combined in vitro, shaped or molded into 2- or 3-dimensional shapes, areas, or volumes, and implanted in situ in the subject. In another aspect, the Fractomer and adipose tissue are a shapeable liquid, semisolid, or molded semisolid prior to administration and following administration, form a solid aggregate. In another aspect, the Fractomer and adipose tissue are co-administered to a subject below the T_{t-heating} of the Fractomer and the Fractomer and adipose tissue form a solid after exposure to the subject's body temperature. In another aspect, the Fractomer permits cell infiltration and vascularization of the adipose tissue.

Another embodiment described herein is a method for preparing an autologous fat graft composition, the method comprising: (a) obtaining adipose tissue from a subject; and (b) combining a recombinant partially ordered polypeptide (Fractomer) with the adipose tissue of step (a) below the T_{t-heating} of the Fractomer to form a mixture. In another aspect, the method further comprises: (c) shaping the mixture into shapes, areas, or volumes. In another aspect, the method further comprises: (d) co-administering the mixture to the subject by injection or implantation. In one aspect, the mixture forms a solid aggregate at a temperature above the T_{t-heating} of the Fractomer.

Another embodiment described herein is a kit comprising a recombinant partially ordered polypeptide (Fractomer), and one or more of containers for combination, molds for specific volumetric dimensions, or a means for adipose tissue aspiration and/or administration.

Another embodiment described herein is the use of a therapeutically effective amount of a recombinant partially ordered polypeptide (Fractomer) and a therapeutically effective amount of adipose tissue for autologous fat grafting in a subject in need thereof.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a proposed method of combining a recombinant partially ordered peptide or “Fractomer” (matrix) with harvested fat (lipoaspirate) for re-injection. Harvested lipoaspirate is combined and mixed with Fractomer solution and the mixture is injected into a deficient area of a subject. Body temperature initiates Fractomer scaffold formation with entrapped lipoaspirate to stabilize the graft.

FIG. 2 shows an example of the structural differences of fat vs lipoaspirate. After liposuction and processing, fat loses its solid-like properties and does not have the mechanical integrity to maintain shape or volume.

FIG. 3A-B show that Fractomer (SEQ ID NO: 12) is thermally responsive and forms a porous network when heated. FIG. 3A shows that at a tunable threshold temperature (e.g., between 20° C. and 37° C.), Fractomer undergoes a phase separation into a porous, solid network (left and middle panels). Three dimensional reconstructed confocal images reveal a complex, elastin-like network when Fractomer is heated (right panel). FIG. 3B shows that the addition of green fluorescent protein (GFP) in a 5% by mass Fractomer solution indicates that the porosity allows greater nutrient flow compared to traditional (e.g., Hyaluronic acid) hydrogels.

FIG. 4A-B show the effect of storage on Fractomer (SEQ ID NO: 12) transition temperature and protein degradation. FIG. 4A shows optical density (OD) measurements of Fractomer resuspended after lyophilization and storage at −20° C. for 1, 2, 5, and 6 months. No change in Fractomer transition temperature was observed at any of the time points. FIG. 4B shows SDS-PAGE analysis of Fractomer resuspended after lyophilization and storage at −20° C. for 1, 5, and 6 months. No changes in Fractomer protein levels or indications of degradation were observed at any of the time points.

FIG. 5A-C show that Fractomer can encapsulate healthy adipocytes and improve shape and projection. FIG. 5A shows confocal microscopy analysis of mixtures of lipoaspirate alone (top panel), 50% by volume of lipoaspirate:Fractomer solution (SEQ ID NO: 12) (250 μM) (middle panels), and 50% by volume of lipoaspirate:Fractomer solution (SEQ ID NO: 12) (750 μM) (bottom panels). Lipoaspirate mixed with different Fractomer concentrations (low=250 μM, high=750 μM) in a 1:1 ratio and stained with LipidTOX (red, also stains Fractomer) and DAPI (blue) indicates the presence of Fractomer around individual adipocyte cells that hold large areas of fat cells together. FIG. 5B shows histological H & E staining analysis of fat alone, and FIG. 5C shows histological H & E staining analysis of lipoaspirate mixed with Fractomer (SEQ ID NO: 18) (250 μM). Fractomer can be seen interspersed between areas of more concentrated fat cells in FIG. 5C.

FIG. 6A-D show example methods and analysis of creating 3D molds using Fractomer alone and fat+Fractomer. FIG. 6A shows an illustration of a mixture of fat:Fractomer (SEQ ID NO: 12) injected into a 3D printed mold and placed at 37° C. to allow Fractomer to aggregate. FIG. 6B shows example molding shapes of Fractomer alone (SEQ ID NO: 12) at low (500 μM, left panel), medium (750 μM, middle panel), and high (2 mM, right panel) concentrations, and indicates increased shape preservation with increased Fractomer concentration. FIG. 6C shows ex vivo molds of fat (human lipoaspirate)+Fractomer solution(SEQ ID NO: 12) mixtures. Mixtures of lipoaspirate with Fractomer solution (750 μM) at different ratios demonstrates that an increased Fractomer:lipoaspirate ratio considerably increases the shape and projection maintenance of the mold compared to fat alone. FIG. 6D shows a comparison between porcine fat alone and a mixture of porcine fat+Fractomer solution (SEQ ID NO: 18) that were injected into 3D printed molds and placed at 37° C. to allow Fractomer to aggregate. Mixtures of porcine lipoaspirate with Fractomer were found to improve shape retention after release from the mold compared to porcine fat alone.

FIG. 7 shows that the shape of Fractomer can be controlled on injection. Mice were injected in the hind flank with infrared labeled Fractomer (SEQ ID NO: 12) in 3 different shape patterns: sphere (left column), dispersed dots (middle column), and elongated rod (right column). IVIS Spectrum imaging at 1 month (top panels) and 4 months (bottom panels) post-injection indicates shape preservation for all 3 injection shape patterns.

FIG. 8A-C show that Fractomer (SEQ ID NO: 12) protein stability and degradation is controllable with concentration. FIG. 8A shows normalized fluorescent intensity of labeled Fractomer at 6 months post-injection in BL/6 mice for different Fractomer solution concentrations (low=250 μM, med=750 μM, high=1500 μM). FIG. 8B shows example fluorescence spectroscopy images of BL/6 mice injected with different Fractomer concentration formulations (low=250 μM, med=750 μM, high=1500 μM) over the course of 6 months. Injections of formulations with high Fractomer concentration were found to persist out to the 6-month time point in situ (bottom row panels). FIG. 8C shows long-term Fractomer resorption profiles post-injection. Normalized fluorescence intensity (left axis) and volume (right axis) were measured for low (250 μM), medium (750 μM), and high (1500 μM) concentrations of Fractomer injections. Injections of formulations with low Fractomer concentration appeared to be fully resorbed after a few months, while injections of formulations with medium and high Fractomer concentrations showed long-term persistence for both fluorescence and volume.

FIG. 9A-F show that Fractomer (SEQ ID NO: 12) improves fat volume retention. FIG. 9A shows an illustration of example fat:Fractomer solution (250 μM) ratios used in experimental groups for injection into nu/nu mice. FIG. 9B shows the effect of fat:Fractomer ratios on volume retention overtime after injection into nu/nu mice. Volume distribution over a 1-week time period shows increased volume retention with the use of Fractomer. FIG. 9C shows a snapshot of the volume distributions at 1 week for each fat:Fractomer solution (Matrix) mixture ratio. FIG. 9D shows the normalized volume at 6 weeks post-injection for different fat:Fractomer mixtures. Volume retention was significantly lower when a reduced fat amount (50% by volume of fat) was used without the inclusion of the stabilizing Fractomer matrix. FIG. 9E shows the volume retention of fat alone and 50% by volume of fat+Fractomer solution (750 μM) injections over a 3-month time period. Fractomer was found to improve fat volume retention over this longer time period. FIG. 9F shows the effective volume of grafted fat between injections of fat alone and injections of fat+Fractomer (750 μM). Per mL of injected fat, the higher concentration Fractomer formulation (750 μM) was found to triple the effective volume of fat alone grafts.

FIG. 10A-C show that the vasculature supply is high within fat grafts made with Fractomer (SEQ ID NO: 12) compared to grafts made of fat alone. FIG. 10A shows histological analysis of fat alone grafts (top panel) versus 1:1 ratio of fat:Fractomer solution (250 μM) grafts (bottom panel) and indicates persistence of Fractomer and a high prevalence of vasculature (CD31+) sections (small arrows) in the supportive Fractomer sections between groups of adipocytes. FIG. 10B shows a histological comparison of fat distribution at 1-month post-injection between fat alone and 1:1 fat:Fractomer solution (250 μM) mixtures. No differences in the cell compositions were observed between the two different graft groups despite 50% by volume of less fat being used in the fat+Fractomer experimental group. FIG. 10C shows a magnified shot of a 1:1 mixture of lipoaspirate and Fractomer solution (250 μM) at 30 days. Solid arrows point to evidence of residual Fractomer and dotted arrows point to newly formed vascular supply within the grafts.

FIG. 11A-B show that cysts form in mice injected with fat alone, while no cyst formation is observed in mice injected with a mixture of fat+Fractomer (SEQ ID NO: 12). FIG. 11A shows cyst formation in mice injected with fat alone over a 3-month time period. Ultrasounds of grafts comprising fat alone or a 1:1 mixture of lipoaspirate and Fractomer solution (750 μM) were captured over 3 months. Arrows point to the development of large oil cysts in the fat alone group (top row, center-right and far-right panels). Greater than 50% of mice injected with fat alone developed cysts, while 0% of mice injected with the mixture of fat+Fractomer formed cysts. FIG. 11B shows a representative histological comparison of cell compositions from fat alone and 1:1 fat:Fractomer solution (750 μM) grafts in mice at 3 months post-injection. The fat alone group showed stark evidence of cyst formation, while no cyst formation was observed in the fat:Fractomer group. Fractomer can be seen supporting adipocytes.

### DETAILED DESCRIPTION

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the terms “effective amount” or “therapeutically effective amount,” refers to a substantially non-toxic, but sufficient amount of an agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human.

As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder, or condition. In some embodiments, the disease, disorder, or condition requires the augmentation of autologous fat grafts. Suitable treatments/conditions include, but are not limited to, radical, post-mastectomy and post-lumpectomy breast reconstruction, cosmetic augmentations, facial reconstructions/implants, rhinoplasty, as well as other treatments requiring the use of autologous fat transfer such as pedal fat pad atrophy, hand reconstruction, Parry-Romberg Syndrome, and craniofacial trauma, and the like.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifest.

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

The term “expression vector” indicates a plasmid, a virus or another medium, known in the art, into which a nucleic acid sequence for encoding a desired protein can be inserted or introduced.

The term “host cell” is a cell that is susceptible to transformation, transfection, transduction, conjugation, and the like with a nucleic acid construct or expression vector.

Host cells can be derived from plants, bacteria, yeast, fungi, insects, animals, etc. In some embodiments, the host cell includes Escherichia coli.

The terms “control,” “reference level,” and “reference” are used herein interchangeably.

The reference level may be a predetermined value or range, which is employed as a benchmark against which to assess the measured result. “Control group” as used herein refers to a group of control subjects. The predetermined level may be a cutoff value from a control group. The predetermined level may be an average from a control group. Cutoff values (or predetermined cutoff values) may be determined by Adaptive index Model (AIM) methodology. Cutoff values (or predetermined cutoff values) may be determined by a receiver operating curve (ROC) analysis from biological samples of the patient group. ROC analysis, as generally known in the biological arts, is a determination of the ability of a test to discriminate one condition from another, e.g., to determine the performance of each marker in identifying a patient having CRC. Alternatively, cutoff values may be determined by a quartile analysis of biological samples of a patient group. For example, a cutoff value may be determined by selecting a value that corresponds to any value in the 25^{th}-75^{th} percentile range, preferably a value that corresponds to the 25^{th} percentile, the 50^{th} percentile or the 75^{th} percentile, and more preferably the 75^{th} percentile. Such statistical analyses may be performed using any method known in the art and can be implemented through any number of commercially available software packages. The healthy or normal levels or ranges for a target or for a protein activity may be defined in accordance with standard practice.

A control may be an agent or cell without a recombinant partially ordered polypeptide (Fractomer). A control may be a molecule, or sample comprising a molecule, with a polypeptide or polymer, that is different from a Fractomer as detailed herein, conjugated thereof, or encapsulated within. A control may be a subject, or a sample therefrom, whose disease state is known. The subject, or sample therefrom, may be healthy, diseased, diseased prior to treatment, diseased during treatment, or diseased after treatment, or a combination thereof. The control may include, for example, an agent or cell alone or by itself.

“Polynucleotide” as used herein can be single stranded or double stranded or can contain portions of both double stranded and single stranded sequence. The polynucleotide can be nucleic acid, natural or synthetic, DNA, genomic DNA, cDNA, RNA, or a hybrid, where the polynucleotide can contain combinations of deoxyribo- and ribo-nucleotides, and combinations of bases including uracil, adenine, thymine, cytosine, guanine, inosine, xanthine hypoxanthine, isocytosine, and isoguanine. Polynucleotides can be obtained by chemical synthesis methods or by recombinant methods.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The terms “polypeptide,” “protein,” and “peptide” are used interchangeably herein. “Primary structure” refers to the amino acid sequence of a particular peptide. “Secondary structure” refers to locally ordered, three dimensional structures within a polypeptide. These structures are commonly known as domains, e.g., enzymatic domains, extracellular domains, transmembrane domains, pore domains, and cytoplasmic tall domains, “Domains” are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three-dimensional structure of a polypeptide monomer. “Quaternary structure” refers to the three-dimensional structure formed by the noncovalent association of independent tertiary units. A “motif” is a portion of a polypeptide sequence and includes at least two amino acids. A motif may be 2 to 20, 2 to 15, or 2 to 10 amino acids in length, in some embodiments, a motif includes 3, 4, 5, 6, or 7 sequential amino acids. A domain may be comprised of a series of motifs, which may be similar or different.

“Recombinant” when used with reference, e.g., to a cell, or nucleic acid, protein, or vector, indicates that the cell, nucleic acid, protein, or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed, or not expressed at all.

“Sample” or “test sample” as used herein can mean any sample in which the presence and/or level of a target is to be detected or determined or any sample comprising an agent, cell, or Fractomer as described herein. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

“Substantially identical” can mean that a first and second amino acid sequence are at least 60%, 65%, 70%, 75%, 80%, 85%, 90%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 amino acids.

“Variant” as used herein with respect to a polynucleotide means (i) a portion or fragment of a referenced nucleotide sequence; (ii) the complement of a referenced nucleotide sequence or portion thereof; (iii) a polynucleotide that is substantially identical to a referenced polynucleotide or the complement thereof; or (iv) a polynucleotide that hybridizes under stringent conditions to the referenced polynucleotide, complement thereof, or a sequences substantially identical thereto.

A “variant” can further be defined as a peptide or polypeptide that differs in amino acid sequence by the insertion, deletion, or conservative substitution of amino acids, but retain at least one biological activity. Representative examples of “biological activity” include the ability to be bound by a specific antibody or polypeptide or to promote an immune response. Variant can mean a substantially identical sequence. Variant can mean a functional fragment thereof. Variant can also mean multiple copies of a polypeptide. The multiple copies can be in tandem or separated by a linker. Variant can also mean a polypeptide with an amino acid sequence that is substantially identical to a referenced polypeptide with an amino acid sequence that retains at least one biological activity. A conservative substitution of an amino acid, i.e., replacing an amino acid with a different amino acid of similar properties (e.g., hydrophilicity, degree, or distribution of charged regions) is recognized in the art as typically involving a minor change. These minor changes can be identified, in part, by considering the hydropathic index of amino acids. See Kyte et al., J. Mol, Biol. 157: 105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indices of ±2 are substituted. The hydrophobicity of amino acids can also be used to reveal substitutions that would result in polypeptides retaining biological function. A consideration of the hydrophilicity of amino acids in the context of a polypeptide permits calculation of the greatest local average hydrophilicity of that polypeptide, a useful measure that has been reported to correlate well with antigenicity and immunogenicity, as discussed in U.S. Pat. No. 4,554,101, which is fully incorporated herein by reference. Substitution of amino acids having similar hydrophilicity values can result in polypeptides retaining biological activity, for example immunogenicity, as is understood in the art. Substitutions can be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant can be a polynucleotide sequence that is substantially identical over the full length of the full gene sequence or a fragment thereof. The polynucleotide sequence can be 80%, 81%, 82%, 83%, 84%, 85%, 88%, 87%, 88%, 89%, 90%, 91%, 92%, 93%, 94%, 95%, 96%, 97%, 98%, 99%, or 100% identical over the full length of the gene sequence or a fragment thereof. A variant can be an amino acid sequence that is substantially identical over the full length of the amino acid sequence or fragment thereof. The amino acid sequence can be 80%, 81%, 82%, 83%, 84%, 85%, 88%, 87%, 88%, 89%, 90%, 91%, 92%, 93%, 94%, 95%, 96%, 97%, 98%, 99%, or 100% identical over the full length of the amino acid sequence or a fragment thereof.

As used herein, “augmented” refers to the improvement of volume, shape, survival, improved blood supply, and reduction in necrosis of autologous adipose tissue or fat grafts. In one aspect, an autologous adipose tissue or fat graft is augmented by the combination of the adipose tissue with a Fractomer which leads to the enhanced long term maintenance and survival of fat graft including conservation of the volume and shape of the fat graft, improved vascularization, enhanced survival, and reduction in necrosis.

As used herein, “concurrently administered” refers to combination of a Fractomer with adipose tissue or lipoaspirate, mixing below the T_{t-heating} point of the Fractomer, administering the combination to the subject, and then upon heating from the subject's body to above the T_{t-heating} point of the Fractomer, the Fractomer surrounds and encapsulates the lipoaspirate in situ.

As used herein, “co-administered” refers to the administration of Fractomer and adipose tissue or lipoaspirate. The co-administration may be “concurrent administration” (as described herein—the combination of the Fractomer and adipose tissue prior to administration) or “sequential administration.” As used herein, “sequential administration” refers to administration where either the Fractomer or adipose tissue are administered first, followed by administration of either the adipose tissue or Fractomer, respectively. In sequence administration, the combination of the Fractomer and adipose tissue occurs in situ following administration.

As used herein, “shapeable” refers to the ability of the Fractomer and adipose tissue composition to be shaped or molded into various two- or three-dimensional shapes, areas, or volumes and the ability to maintain this shape, area, or volume over a prolonged period of time. Specific exemplary shapes or volumes include breasts, buttocks, hands, knees, 2-dimensional layers for placing under skin or skin grafts, or other irregular or indefinite shapes or volumes present in a subject's body.

### Compositions

One embodiment described herein is an injectable tissue matrix composition to augment autologous fat grafts, the composition comprising, consisting of, or consisting essentially of a recombinant partially ordered polypeptide or “Fractomer” and adipose tissue. In some embodiments, the adipose tissue comprises a lipoaspirate. In one embodiment, the composition comprises about 10-90% by volume of lipoaspirate including all integers within the specified range. In another embodiment, the composition comprises about 25-90% by volume of lipoaspirate including all integers within the specified range. In some embodiments, the composition comprises at least 25% by volume of lipoaspirate. In other embodiments, the composition comprises at least 35% by volume of lipoaspirate. In other embodiments, the composition comprises at least 45% by volume of lipoaspirate. In yet other embodiments, the composition comprises at least 50% by volume of lipoaspirate. In other embodiments, the composition comprises at least 60% by volume of lipoaspirate. In other embodiments, the composition comprises at least 70% by volume of lipoaspirate. In other embodiments, the composition comprises at least 80% by volume of lipoaspirate. In other embodiments, the composition comprises at least 90% by volume of lipoaspirate. The volumes of fat or lipoaspirate are combined with solutions of Fractomer at specific concentrations to provide various fat:Fractomer ratios as described herein. As an example, 900 μL of fat or lipoaspirate combined with 100 μL of Fractomer solution would be a 9:1 ratio by volume or 90% fat by volume.

The term “Fractomer” as used herein refers to the class of recombinant, artificial proteins designed to mimic native elastin that are thermally responsive, allowing them to be injected as a liquid, yet rapidly form a porous, solid network at body temperature. Exemplary Fractomers are described in International Patent Application Publication No. WO 2019006374 A1, which is incorporated by reference herein in its entirety. In some embodiments, the Fractomer comprises a recombinant partially ordered polypeptide (POP). Each POP may include a plurality of disordered domains, and a plurality of structured domains. The POP may exhibit phase transition behavior by changing solubility and aggregate dissolution/formation with temperature.

A Fractomer consists of a structured domain of oligoalanine amino acids (from 5 to 500, but typically A₂₅) that form perfect α-helices and are periodically inserted into an unstructured elastin-like polypeptide (ELP) that is composed of typically 80-120 total repeats of a (GXGVP)ₙ pentapeptide motif (˜30-50 kDa) (SEQ ID NO: 1), where X is any standard amino acid except proline. In one aspect, the unstructured polypeptide or “disordered domain” is a (GXGVP)ₙ motif (SEQ ID NO: 2), wherein X is Val (SEQ ID NO: 3), or Ala (SEQ ID NO: 4), or mixture of Ala and Val, and wherein n is an integer from 1 to 50. In one aspect, X is an alternating iteration of Ala and Val in a ratio from 10:1 to 1:10 (Ala:Val). In another aspect, X is an alternating iteration of Ala and Val in a ratio of 1:1 (SEQ ID NO: 5) or 1:4 (SEQ ID NO: 6). Fractomers are recombinantly synthesized in E. coli by overexpression of a plasmid-borne gene that encodes the Fractomer.

Fractomers have several advantages over traditional hydrogels. The ability of Fractomers to spontaneously crosslink upon subcutaneous injection solely via hydrophobic interactions between the α-helical (Ala)₂₅ domains is significant. The majority of chemical crosslinking techniques—required to crosslink elastin-like polypeptides (ELPs) or other synthetic polymers into hydrogels—have significant disadvantages. Small molecule crosslinkers can often be toxic, and it is difficult to control the kinetics of chemical or enzymatic crosslinking of an injected polymer solution in situ. Moreover, most hydrogels are either non-porous or require the use of porogens or templating methods to introduce the porosity necessary for in vivo material integration. These materials must be subsequently implanted at the desired site. While these obstacles can be overcome, the simplicity of physical crosslinking and the spontaneous formation of a mechanically stable, porous network under the simple action of body heat is an unprecedented advantage for the use of Fractomers in fat grafting compared to other injectable materials.

The disordered domains and the structured domains of the Fractomer can be arranged in any number of possible ways, in some embodiments, one or more disordered domains are positioned between at least two adjacent structured domains of the Fractomer. In some embodiments, the Fractomer includes a plurality of structured domains repeated in tandem and a plurality of disordered domains repeated in tandem, in some embodiments, the plurality of structured domains repeated in tandem are positioned C-terminal to the plurality of disordered domains repeated in tandem, in some embodiments, the plurality of structured domains repeated in tandem are positioned N-terminal to the plurality of disordered domains repeated in tandem. In some embodiments, the Fractomer is arranged as [disordered domain]q-[structured domain]r[disordered domain]s-[structured domain]t, wherein q, r, s, and t are independently an integer from 0 to 100, such as from 1 to 100, from 2 to 100, from 1 to 50 or from 2 to 50. In some embodiments, the Fractomer is arranged as [disordered domain]q-[structured domain]r, wherein q and r are independently an integer from 1 to 100. in some embodiments, q, r, s, and t are independently an integer from 0 to 10, from 0 to 20, from 0 to 30, from 0 to 40, from 0 to 50, from 0 to 60, from 0 to 70, from 0 to 80, from 0 to 90, from 0 to 100, from 1 to 10, from 1 to 20, from 1 to 30, from 1 to 40, from 1 to 150, from 1 to 60, from 1 to 70, from 1 to 80, from 1 to 90 or from 1 to 100.

The Fractomer may include a plurality of disordered domains. The disordered domain may comprise any polypeptide that has minimal or no secondary structure as observed by CD and have phase transition behavior. The disordered domain may include an amino acid sequence of repeated amino acids, non-repeated amino acids, or a combination thereof.

In some embodiments, about 20% to about 99%, such as about 25% to about 97%, about 35% to about 95% or about 50% to about 94% of the Fractomer comprises disordered domains. At least about 2%, 3%, 4%, 5%, 10%, 15%, 20%, 25%, 30%, 35%, 40%, 45%, 50%, 55%, 60%, 65%, 70%, 75%, 80%, 85%, 90%, or 95% of the Fractomer may comprise disordered domains.

In some embodiments, the disordered domain comprises an amino acid sequence of (GXGVP)ₙ (SEQ ID NO:1), wherein X is any amino acid and n is an integer greater than or equal to 1. In some embodiments, m is an integer from 1 to 500. In some embodiments, m is at least, at most, or exactly 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, or 500. in some embodiments, m may be less than 500, less than 400, less than 300, less than 200, or less than 100. In some embodiments, m is from 1 to 500, from 1 to 400, from 1 to 300, from 1 to 200, or from 60 to 180. In some embodiments, m is 60, 120, or 180. In some embodiments, X is any amino acid except proline. In some embodiments, X is Val, or Ala, or an alternating iteration of Ala and Val. In some embodiments, X is Val. In some embodiments, X is Ala. in some embodiments, X is an alternating iteration of Ala and Val. In some embodiments, X is an alternating iteration of Ala and Val in a ratio of 1:1, 1:2, 1:3, 1:4, 1:5, 1:6, 1:7, 1:8, 1:9, 1:10, 2:1, 3:1, 4:1, 5:1, 6:1, 7:1, 8:1, 9:1, or 10:1. In some embodiments, X is a mixture of Ala and Val in a ratio of 1:1 or 1:4. In some embodiments, X is an alternating iteration of Ala and Val in a ratio from 10:1 to 1:10 (Ala:Val), such as from 5:1 to 1:5 or from 1:1 to 1:4.

### Structured Domains

The Fractomer may include a plurality of structured domains. The structured domain may have a secondary structure as observed by CD, such as, for example, an alpha helix. The structured domain may comprise at least one of a polyproline domain and a polyalanine domain, in some embodiments, the Fractomer comprises alternating disordered domains and structured domains. In some embodiments, the structured domain comprises only polyalanine domains. In some embodiments, the structured domain comprises only polyproline domains.

In some embodiments, about 4% to about 75%, such as about 5% to about 70%, about 6% to about 60% or about 7% to about 50% of the Fractomer comprises structured domains. At least about 2%, 3%, 4%, 5%, 10%, 15%, 20%, 25%, 30%, 35%, 40%, 45%, 50%, 55%, 60%, 65%, 70%, 75%, 80%, 85%, 90%, or 95% of the Fractomer may comprise structured domains.

In some embodiments, the structured domain comprises a polyalanine domain. Each polyalanine domain may include at least 5 alanine residues. Each polyalanine domain may have at least about 60% of the amino acids in an alpha-helical conformation. In one aspect, the structured domain comprises a polyalanine domain (Ala)ₘ wherein m is an integer from 5 to 500. In another aspect, the polyalanine domain comprises of one or more of: (A)₂₅ (SEQ ID NO: 7); K(A)₂₅K (SEQ ID NO: 8); D(A)₂₅K (SEQ ID NO: 9); GD(A₂₅)K (SEQ ID NO: 10); or GK(A₂₅)K (SEQ ID NO: 11).

In one embodiment, the Fractomer comprises a plurality of disordered domains, each comprising a PG motif comprising an amino acid sequence selected from PG, P(X)ₙG (SEQ ID NO: 20), and (B)ₘP(X)ₙG(Z)ₚ (SEQ ID NO: 21), or a combination thereof, wherein m, n, and p are independently an integer from 1 to 15, and wherein B, X, and Z are independently any amino acid; and a plurality of structured domains, each comprising a polyalanine domain, each polyalanine domain comprising at least 5 alanine residues and having at least about 50% of the amino acids in an α-helical conformation; wherein the Fractomer exhibits phase transition behavior. In one aspect, at least one disordered domain comprises an amino acid sequence of (GXGVP)ₙ (SEQ ID NO:1), wherein X is any amino acid except proline and n is an integer greater than or equal to 1. In another aspect, at least about 60% of the amino acids in each polyalanine domain are in an α-helical conformation. In another aspect, each polyalanine domain comprises an amino acid sequence of [Bₚ(A)_{q}Zᵣ]ₙ (SEQ ID NO: 22) or [(BAₛ)ₜZr]ₙ (SEQ ID NO: 23), wherein B is Lys, Arg, Asp, or Glu; A is Ala; Z is Lys, Arg, Asp, or Glu; n is an integer from 1 to 50; p is an integer from 0 to 2; q is an integer from 1 to 50; r is an integer from 0 to 2; s is an integer from 1 to 5; and t is an integer from 1 to 50. In another aspect, the structured domain comprises one or more of (A)₂₅ (SEQ ID NO: 7); K(A)₂₅K (SEQ ID NO: 8); D(A)₂₅K (SEQ ID NO: 9); GD(A₂₅)K (SEQ ID NO: 10); or GK(A₂₅)K (SEQ ID NO: 11). In another aspect, about 4% to about 75% of the Fractomer comprises structured domains. In another aspect, the Fractomer is soluble below a lower critical solution temperature (LCST). In another aspect, the Fractomer has a transition temperature of heating (T_{t-heating}) and a transition temperature of cooling (T_{t-cooling}), and wherein the transition temperature of heating (T_{t-heating}) and transition temperature of cooling (T_{t-cooling}) are identical, or wherein the transition temperature of heating (T_{t-heating}) is greater than the transition temperature of cooling (T_{t-cooling}).

In another embodiment, the Fractomer comprises a plurality of disordered domains; and a plurality of structured domains. In one aspect, the Fractomer has the general structure of [(GXGVP)ₙ-α-helix]ₘ, where X can be any amino acid except proline and α-helix is any polyalanine based α-helix having about 5 to 50 Alanine residues. In another aspect, the Fractomer has the structure [(GXGVP)ₙ-GX¹(A)₂₅X¹]ₘ; where X is A or V; X¹ is K or D; n is an integer from 10 to 20; and m is an integer from 4 to 8 (e.g., [(SEQ ID NO: 2)ₙ-(SEQ ID NO: 10 or 11)]ₘ). In another aspect, the Fractomer comprises one or more of the following structures:

(SEQ ID NO: 12)
M[(GVGVP)₁₅-GD(A25)K]₆-GWP;
(SEQ ID NO: 13)
M[(GVGVP)₁₅-GD(A25)K]₄-GWP;
(SEQ ID NO: 14)
M[(GVGVP)₁₅-GK(A25)K]₆-GWP;
(SEQ ID NO: 15)
M[(GVGVP)₁₅-GK(A25)K]₄-GWP;
(SEQ ID NO: 16)
M[(G[A1:V1]GVP)₆-GD(A₂₅)K]₆-GWP;
(SEQ ID NO: 17)
M[(G[A1:V1]GVP)₁₆-GD(A₂₅)K]₄-GWP;
(SEQ ID NO: 18)
M[(G[V4:A1]GVP)₁₅-GD(A₂₅)K]₆-GWP;
or
(SEQ ID NO: 19)
M[(G[V4:A1]GVP)₁₅-GD(A₂₅)K]₄-GWP.

In one aspect, the Fractomer comprises the following structures:

(SEQ ID NO: 12)
M[(GVGVP)₁₅-GD(A₂₅)K]₆-GWP;
or
(SEQ ID NO: 18)
M[(G[V4:A1]GVP)₁₅-GD(A₂₅)K]₆-GWP.

Exemplary sequences, sequence motifs, and Fractomer constructs as described herein are shown below in Table 1.

TABLE 1
Exemplary Sequences of Motifs and Fractomer Constructs

GXGVP (SEQ ID NO: 1)
GXGVP
X is any amino acid except proline
Sequence can be repeated 1 or more times
GXGVP (SEQ ID NO: 2)
GXGVP
Xaa is Ala or Val
Sequence can be repeated 1 or more times
GAGVP (SEQ ID NO: 3)
GAGVP
Sequence can be repeated 1 or more times
GVGVP (SEQ ID NO: 4)
GVGVP
Sequence can be repeated 1 or more times
G[A1:V1]GVP (SEQ ID NO: 5)
GVGVPGAGVP
G[A1:V1]GVP = GAVGPGVGVP; repeats shown; Ala in first repeat; Val in second repeat; this motif
can be repeated 1 or more times
[G[V4:A1]GVP] (SEQ ID NO: 6)
GVGVPGVGVPGVGVPGVGVPGAGVP
G[V4:A1]GVP = GVGVPGAGVP; 5 repeats shown; Val in the first 4 repeats and Ala in the 5^{th} repeat;
this motif can be repeated 1 or more times
(A)₂₅ (SEQ ID NO: 7)
AAAAAAAAAAAAAAAAAAAAAAAAA
K(A)₂₅K (SEQ ID NO: 8)
KAAAAAAAAAAAAAAAAAAAAAAAAAK
D(A)₂₅K (SEQ ID NO: 9)
DAAAAAAAAAAAAAAAAAAAAAAAAAK
GD(A₂₅)K (SEQ ID NO: 10)
GDAAAAAAAAAAAAAAAAAAAAAAAAAK
GK(A₂₅)K (SEQ ID NO: 11)
GKAAAAAAAAAAAAAAAAAAAAAAAAAK
M[(GVGVP)₁₅-GD(A₂₅)K]₆-GWP (SEQ ID NO: 12)
MGVGVPGVGVPGVGVPGVGVPGVGVPGVGVPGVGVPGVGVPGVGVPGVGVPGVGVPGVGVPGVGVPGVGVPGVGVP
GDAAAAAAAAAAAAAAAAAAAAAAAAAKGVGVPGVGVPGVGVPGVGVPGVGVPGVGVPGVGVPGVGVPGVGVPGVG
VPGVGVPGVGVPGVGVPGVGVPGVGVPGDAAAAAAAAAAAAAAAAAAAAAAAAAKGVGVPGVGVPGVGVPGVGVPG
VGVPGVGVPGVGVPGVGVPGVGVPGVGVPGVGVPGVGVPGVGVPGVGVPGVGVPGDAAAAAAAAAAAAAAAAAAAA
AAAAAKGVGVPGVGVPGVGVPGVGVPGVGVPGVGVPGVGVPGVGVPGVGVPGVGVPGVGVPGVGVPGVGVPGVGVP
GVGVPGDAAAAAAAAAAAAAAAAAAAAAAAAAKGVGVPGVGVPGVGVPGVGVPGVGVPGVGVPGVGVPGVGVPGVG
VPGVGVPGVGVPGVGVPGVGVPGVGVPGVGVPGDAAAAAAAAAAAAAAAAAAAAAAAAAKGVGVPGVGVPGVGVPG
VGVPGVGVPGVGVPGVGVPGVGVPGVGVPGVGVPGVGVPGVGVPGVGVPGVGVPGVGVPGDAAAAAAAAAAAAAAA
AAAAAAAAAAKGWP
M[(GVGVP)₁₅-GD(A₂₅)K]₄-GWP (SEQ ID NO: 13)
MGVGVPGVGVPGVGVPGVGVPGVGVPGVGVPGVGVPGVGVPGVGVPGVGVPGVGVPGVGVPGVGVPGVGVPGVGVP
GDAAAAAAAAAAAAAAAAAAAAAAAAAKGVGVPGVGVPGVGVPGVGVPGVGVPGVGVPGVGVPGVGVPGVGVPGVG
VPGVGVPGVGVPGVGVPGVGVPGVGVPGDAAAAAAAAAAAAAAAAAAAAAAAAAKGVGVPGVGVPGVGVPGVGVPG
VGVPGVGVPGVGVPGVGVPGVGVPGVGVPGVGVPGVGVPGVGVPGVGVPGVGVPGDAAAAAAAAAAAAAAAAAAAA
AAAAAKGVGVPGVGVPGVGVPGVGVPGVGVPGVGVPGVGVPGVGVPGVGVPGVGVPGVGVPGVGVPGVGVPGVGVP
GVGVPGDAAAAAAAAAAAAAAAAAAAAAAAAAKGWP
M[(GVGVP)₁₅-GK(A₂₅)K]₆-GWP (SEQ ID NO: 14)
MGVGVPGVGVPGVGVPGVGVPGVGVPGVGVPGVGVPGVGVPGVGVPGVGVPGVGVPGVGVPGVGVPGVGVPGVGVP
GKAAAAAAAAAAAAAAAAAAAAAAAAAKGVGVPGVGVPGVGVPGVGVPGVGVPGVGVPGVGVPGVGVPGVGVPGVG
VPGVGVPGVGVPGVGVPGVGVPGVGVPGKAAAAAAAAAAAAAAAAAAAAAAAAAKGVGVPGVGVPGVGVPGVGVPG
VGVPGVGVPGVGVPGVGVPGVGVPGVGVPGVGVPGVGVPGVGVPGVGVPGVGVPGKAAAAAAAAAAAAAAAAAAAA
AAAAAKGVGVPGVGVPGVGVPGVGVPGVGVPGVGVPGVGVPGVGVPGVGVPGVGVPGVGVPGVGVPGVGVPGVGVP
GVGVPGKAAAAAAAAAAAAAAAAAAAAAAAAAKGVGVPGVGVPGVGVPGVGVPGVGVPGVGVPGVGVPGVGVPGVG
VPGVGVPGVGVPGVGVPGVGVPGVGVPGVGVPGKAAAAAAAAAAAAAAAAAAAAAAAAAKGVGVPGVGVPGVGVPG
VGVPGVGVPGVGVPGVGVPGVGVPGVGVPGVGVPGVGVPGVGVPGVGVPGVGVPGVGVPGKAAAAAAAAAAAAAAA
AAAAAAAAAAKGWP
M[(GVGVP)₁₅-GK(A₂₅)K]₄-GWP (SEQ ID NO: 15)
MGVGVPGVGVPGVGVPGVGVPGVGVPGVGVPGVGVPGVGVPGVGVPGVGVPGVGVPGVGVPGVGVPGVGVPGVGVP
GKAAAAAAAAAAAAAAAAAAAAAAAAAKGVGVPGVGVPGVGVPGVGVPGVGVPGVGVPGVGVPGVGVPGVGVPGVG
VPGVGVPGVGVPGVGVPGVGVPGVGVPGKAAAAAAAAAAAAAAAAAAAAAAAAAKGVGVPGVGVPGVGVPGVGVPG
VGVPGVGVPGVGVPGVGVPGVGVPGVGVPGVGVPGVGVPGVGVPGVGVPGVGVPGKAAAAAAAAAAAAAAAAAAAA
AAAAAKGVGVPGVGVPGVGVPGVGVPGVGVPGVGVPGVGVPGVGVPGVGVPGVGVPGVGVPGVGVPGVGVPGVGVP
GVGVPGKAAAAAAAAAAAAAAAAAAAAAAAAAKGWP
M[(G[A1:V1]GVP)₁₆-GD(A₂₅)K]₆-GWP (SEQ ID NO: 16)
MGAGVPGVGVPGAGVPGVGVPGAGVPGVGVPGAGVPGVGVPGAGVPGVGVPGAGVPGVGVPGAGVPGVGVPGAGVP
GVGVPGDAAAAAAAAAAAAAAAAAAAAAAAAAKGAGVPGVGVPGAGVPGVGVPGAGVPGVGVPGAGVPGVGVPGAG
VPGVGVPGAGVPGVGVPGAGVPGVGVPGAGVPGVGVPGDAAAAAAAAAAAAAAAAAAAAAAAAAKGAGVPGVGVPG
AGVPGVGVPGAGVPGVGVPGAGVPGVGVPGAGVPGVGVPGAGVPGVGVPGAGVPGVGVPGAGVPGVGVPGDAAAAA
AAAAAAAAAAAAAAAAAAAAKGAGVPGVGVPGAGVPGVGVPGAGVPGVGVPGAGVPGVGVPGAGVPGVGVPGAGVP
GVGVPGAGVPGVGVPGAGVPGVGVPGDAAAAAAAAAAAAAAAAAAAAAAAAAKGAGVPGVGVPGAGVPGVGVPGAG
VPGVGVPGAGVPGVGVPGAGVPGVGVPGAGVPGVGVPGAGVPGVGVPGAGVPGVGVPGDAAAAAAAAAAAAAAAAA
AAAAAAAAKGAGVPGVGVPGAGVPGVGVPGAGVPGVGVPGAGVPGVGVPGAGVPGVGVPGAGVPGVGVPGAGVPGV
GVPGAGVPGVGVPGDAAAAAAAAAAAAAAAAAAAAAAAAAKGWP
M[(G[A1:V1]GVP)₁₆-GD(A₂₅)K]₄-GWP (SEQ ID NO: 17)
MGAGVPGVGVPGAGVPGVGVPGAGVPGVGVPGAGVPGVGVPGAGVPGVGVPGAGVPGVGVPGAGVPGVGVPGAGVP
GVGVPGDAAAAAAAAAAAAAAAAAAAAAAAAAKGAGVPGVGVPGAGVPGVGVPGAGVPGVGVPGAGVPGVGVPGAG
VPGVGVPGAGVPGVGVPGAGVPGVGVPGAGVPGVGVPGDAAAAAAAAAAAAAAAAAAAAAAAAAKGAGVPGVGVPG
AGVPGVGVPGAGVPGVGVPGAGVPGVGVPGAGVPGVGVPGAGVPGVGVPGAGVPGVGVPGAGVPGVGVPGDAAAAA
AAAAAAAAAAAAAAAAAAAAKGAGVPGVGVPGAGVPGVGVPGAGVPGVGVPGAGVPGVGVPGAGVPGVGVPGAGVP
GVGVPGAGVPGVGVPGAGVPGVGVPGDAAAAAAAAAAAAAAAAAAAAAAAAAKGWP
M[(G[V4:A1]GVP)₁₅-GD(A₂₅)K]₆-GWP (SEQ ID NO: 18)
MGVGVPGVGVPGVGVPGVGVPGAGVPGVGVPGVGVPGVGVPGVGVPGAGVPGVGVPGVGVPGVGVPGVGVPGAGVP
GDAAAAAAAAAAAAAAAAAAAAAAAAAKGVGVPGVGVPGVGVPGVGVPGAGVPGVGVPGVGVPGVGVPGVGVPGAG
VPGVGVPGVGVPGVGVPGVGVPGAGVPGDAAAAAAAAAAAAAAAAAAAAAAAAAKGVGVPGVGVPGVGVPGVGVPG
AGVPGVGVPGVGVPGVGVPGVGVPGAGVPGVGVPGVGVPGVGVPGVGVPGAGVPGDAAAAAAAAAAAAAAAAAAAA
AAAAAKGVGVPGVGVPGVGVPGVGVPGAGVPGVGVPGVGVPGVGVPGVGVPGAGVPGVGVPGVGVPGVGVPGVGVP
GAGVPGDAAAAAAAAAAAAAAAAAAAAAAAAAKGVGVPGVGVPGVGVPGVGVPGAGVPGVGVPGVGVPGVGVPGVG
VPGAGVPGVGVPGVGVPGVGVPGVGVPGAGVPGDAAAAAAAAAAAAAAAAAAAAAAAAAKGVGVPGVGVPGVGVPG
VGVPGAGVPGVGVPGVGVPGVGVPGVGVPGAGVPGVGVPGVGVPGVGVPGVGVPGAGVPGDAAAAAAAAAAAAAAA
AAAAAAAAAAKGWP
M[(G[V4:A1]GVP)₁₅-GD(A₂₅)K]₄-GWP (SEQ ID NO: 19)
MGVGVPGVGVPGVGVPGVGVPGAGVPGVGVPGVGVPGVGVPGVGVPGAGVPGVGVPGVGVPGVGVPGVGVPGAGVP
GDAAAAAAAAAAAAAAAAAAAAAAAAAKGVGVPGVGVPGVGVPGVGVPGAGVPGVGVPGVGVPGVGVPGVGVPGAG
VPGVGVPGVGVPGVGVPGVGVPGAGVPGDAAAAAAAAAAAAAAAAAAAAAAAAAKGVGVPGVGVPGVGVPGVGVPG
AGVPGVGVPGVGVPGVGVPGVGVPGAGVPGVGVPGVGVPGVGVPGVGVPGAGVPGDAAAAAAAAAAAAAAAAAAAA
AAAAAKGVGVPGVGVPGVGVPGVGVPGAGVPGVGVPGVGVPGVGVPGVGVPGAGVPGVGVPGVGVPGVGVPGVGVP
GAGVPGDAAAAAAAAAAAAAAAAAAAAAAAAAKGWP
P(X)ₙG (SEQ ID NO: 20)
PXXXXXXXXXXXXXXXG
X is any amino acid and any one or all of amino acids 3-16 can be either present or absent
(B)ₘP(X)ₙG(Z)ₚ (SEQ ID NO: 21)
BBBBBBBBBBBBBBBPXXXXXXXXXXXXXXXGZZZZZZZZZZZZZZZ
B, X, and Z are independently any amino acid; and a plurality of structured domains, each comprising
a polyalanine domain, each polyalanine domain comprising at least 5 alanine residues and having at
least about 50% of the amino acids in an α-helical conformation
[Bₚ(A)_{q}Zᵣ]ₙ (SEQ ID NO: 22)
BBAAAAAAAAAAAAAAAAAAAAAAAAAAAAAAAAAAAAAAAAAAAAAAAAAAZZ
B is Lys, Arg, Asp, or Glu; A is Ala; Z is Lys, Arg, Asp, or Glu; n is an integer from 1 to 50; p is
an integer from 0 to 2; q is an integer from 1 to 50; r is an integer from 0 to 2; s is an integer
from 1 to 5; and t is an integer from 1 to 50
[(BAₛ)Zᵣ]ₙ (SEQ ID NO: 23)
BAZ
B is Lys, Arg, Asp, or Glu; A is Ala; Z is Lys, Arg, Asp, or Glu; n is an integer from 1 to 50; p
is an integer from 0 to 2; q is an integer from 1 to 50; r is an integer from 0 to 2; s is an integer
from 1 to 5; and t is an integer from 1 to 50

The Fractomer may also include amino acid derivatives that are not naturally occurring, such as a UV crosslinkable amino acid derivative. The non-native amino acid derivative can be used to introduce covalent crosslinks between different Fractomers and within the same Fractomer. For example, Fractomers that include the UV crosslinkable amino acid derivative can be exposed to UV light, which can result in covalent crosslinks being formed between the amino acid derivative and a side chain of an amino acid of another Fractomer or with a side chain of an amino acid of the same Fractomer (having the amino acid derivative). The UV crosslinkable amino acid derivative may be any amino acid that has been functionalized with an azide group. In some embodiments, the amino acid derivative is para-azidophenylalanine.

The UV crosslinkable amino acid derivative may be included at varying amounts without affecting the Fractomer's ability to transition at different temperatures. For example, the UV crosslinkable amino acid derivative may be included within the Fractomer from about 0.1% to about 20% (of the Fractomer), such as from about 0.5% to about 15% or from about 1% to about 10% (of the Fractomer).

The Fractomer may demonstrate phase transition behavior by changing solubility and aggregate formation with temperature. The phase transition behavior of the Fractomer may derive from the phase transition behavior of the disordered domains of the Fractomer. “Phase transition” or “transition” may refer to the aggregation of a polypeptide, which occurs sharply at a specific temperature. The phase transition may be reversible, although the specific temperature of dissolution may be the same or different from the specific temperature of aggregation.

In some embodiments, the Fractomer is soluble below a lower critical solution temperature (LCST). LCST is the temperature below which the polypeptide is miscible.

A transition temperature (Tₜ) is a temperature at which the Fractomer changes from one state to another. States may include, for example, soluble polypeptides, gels, and aggregates of varying sizes and dimensions. The Fractomer may have a transition temperature of heating (T_{t-heating}) and a transition temperature of cooling (T_{t-cooling}). In some embodiments, the transition temperature heating (T_{t-heating}) is concentration-dependent. In some embodiments, the transition temperature cooling (T_{t-cooling}) is concentration-independent. The T_{t-heating} may be primarily determined by the disordered domains. The T_{t-cooling} may be primarily determined by the structured domains.

Below the transition temperature (LCST or Tₜ), the Fractomer may be highly soluble. Upon heating above the transition temperature, the Fractomer may hydrophobically collapse and aggregate, forming a separate phase.

The Fractomer may phase transition at a variety of temperatures. The Fractomer may have a transition temperature (Tₜ) from about 0° C. to about 100° C., from about 10° C. to about 50° C., or from about 20° C. to about 42° C. The transition temperature of heating (T_{t-heating}) and transition temperature of cooling (T_{t-cooling}) may be identical. As used herein, temperatures may be “identical” when the temperatures are within 2.0° C., 1.0° C., 0.5° C., or 0.1° C. of each other. In some embodiments, the transition temperature of heating (T_{t-heating}) is greater than the transition temperature of cooling (T_{t-cooling}). In embodiments where the Fractomer has a T_{t-heating} greater than the T_{t-cooling}, the difference between the two transition temperatures may be referred to as a hysteresis, in some embodiments, the Fractomer has a hysteresis of about 5° C. to about 70° C., such as about 5° C. to about 60° C. or about 10° C. to about 50° C.

The phase transition behavior of the Fractomer may be utilized in purification of the Fractomer according to a method referred to as “inverse transition cycling,” in which the Fractomer's reversible phase transition behavior is used to cycle the solution through soluble and insoluble phases, thereby removing contaminants. Phase transition may also be triggered using kosmotropic salts, such as, for example, ammonium sulfate or sodium chloride. The kosmotropic salt may be added to a solution comprising the Fractomer, with the kosmotropic salt being added until the Fractomer forms aggregates or is precipitated out of solution. The aggregates may be pelleted by centrifugation and resuspended in a second solution or buffer. Aggregates of the Fractomer may re-solubilize into solution once cooled below their Tₜ or when the kosmotropic salt is removed from the solution. In some embodiments, the Fractomer is purified without any chromatographic purification. In some embodiments, the Fractomer is generated recombinantly and purified from bacterial culture, such as, for example, from E. coli.

In some embodiments, the Fractomer may form an aggregate when the temperature is greater than the T_{t-heating}. The aggregate may resolubilize when cooled to below a temperature less than the T_{t-cooling}.

The aggregate formed by a plurality of Fractomers may have advantageous properties that can arise from the structure of the Fractomers. For example, the aggregate may have physical, non-covalent crosslinks. These physical, non-covalent crosslinks may arise from helical bundling of the structured domain(s) interacting with each other. The aggregate may also have covalent crosslinks (e.g., chemical crosslinks) in addition to physical, non-covalent crosslinks. Covalent crosslinks can be included in the aggregate in order to increase their mechanical stability without altering their porous architecture, in some embodiments, the aggregate can be formed from a plurality of Fractomers and can then be further stabilized by covalent crosslinking (after the formation of the aggregate). Covalent crosslinks can be introduced via a UV crosslinkable amino acid derivative having an azide functionality as described herein. Further examples of crosslinks that can be incorporated into the aggregate include, but are not limited to, small molecule crosslinks and cysteine disulfide bridges. An example of a chemical, small molecule crosslink is tetrakis(hydroxymethyl)phosphonium chloride (TMPC), which can crosslink lysines within Fractomers.

In addition, the aggregate formed by a plurality of Fractomers may have solid-like properties that distinguish it from liquid-like coacervate structures. For example, the aggregate may have a storage modulus (C) that is greater than its loss modulus (G″), such as having a G′ 2× greater, 5× greater, 10× greater, 15× greater, 20× greater, 25× greater, 30× greater, 35× greater, 50× greater or 100× greater than its G″. In some embodiments, the aggregate has a G′ from 2× greater to 100× greater than its G″, such as from 10× greater to 50× greater or from 20× greater to 35× greater than its G″.

The aggregate formed from a plurality of Fractomers may be a variety of sizes and dimensions. In some embodiments, the aggregate is a stable three-dimensional matrix. In some embodiments, the aggregate is fractal-like, in some embodiments, the aggregate is gel-like, in some embodiments, the aggregate is porous with a void volume, e.g., the nonprotein rich phase of the aggregate. In some embodiments, the void volume is tunable. For example, the aggregate may have a void volume from about 60% to about 90% (of the volume of the aggregate), in addition, the aggregate may comprise pores having a diameter of about 1 μm to about 100 μm, such as about 1 μm to about 10 μm, about 3 μm to about 5 μm, about 25 μm to about 60 μm, about 30 μm to about 50 μm, or about 3 μm to about 50 μm.

Further provided are polynucleotides encoding the Fractomers described herein. A vector may include the polynucleotide encoding the Fractomers detailed herein. To obtain expression of a polypeptide, one may subclone the polynucleotide encoding the polypeptide into an expression vector that contains a promoter to direct transcription, a transcription/translation terminator, and if for a nucleic acid encoding a protein, a ribosome binding site for translational initiation. An example of a vector is pET24. Suitable bacterial promoters are well known in the art. Further provided is a host cell transformed or transfected with an expression vector comprising a polynucleotide encoding a Fractomer as described herein. Bacterial expression systems for expressing the protein are available in, e.g., E. coli, Bacillus sp., and Salmonella. See Paiva et al., Gene 22: 229-235 (1983); Mosbach et al., Nature 302: 543-545 (1983). Kits for such expression systems are commercially available. Eukaryotic expression systems for mammalian cells, yeast, and insect cells are well known in the art and are also commercially available. Retroviral expression systems can be used in the present invention.

The Fractomer may be expressed recombinantly in a host cell according to one of skill in the art. The Fractomer may be purified by any means known to one of skill in the art. For example, the Fractomer may be purified using chromatography, such as liquid chromatography, size exclusion chromatography, or affinity chromatography, or a combination thereof, in some embodiments, the Fractomer is purified without chromatography, in some embodiments, the Fractomer is purified using inverse transition cycling.

In other embodiments, the present disclosure further provides herein a scaffold comprising a plurality of Fractomers. The scaffold may be formed at a temperature greater than the transition temperature of the Fractomer, such that the polypeptide forms an aggregate. The scaffold may be injectable.

Further provided in accordance with one embodiment is a cellular scaffold. A cellular scaffold includes the scaffold and a plurality of cells. The cells may include a variety of types. In some embodiments, the cells comprise stem cells, bacterial cells, or human tissue cells, or a combination thereof.

The scaffold may have low immunogenicity or low antigenicity or both. The scaffold may promote at least one of cell growth, cell recruitment, and cell differentiation, or a combination thereof. The scaffold, or cellular scaffold, may be suitable for cell transplantation, tissue regeneration, cell culture, and cell-based in vitro assays. In addition, the scaffold and/or cellular scaffold may promote the formation of vasculature, wound healing, or a combination thereof.

Further provided in accordance with one embodiment is a drug delivery composition. The drug delivery composition may include a plurality of Fractomers as detailed herein, self-assembled into an aggregate above the T_{t-heating}, and an agent encapsulated within the aggregate.

The agent may be a therapeutic. In some embodiments, the agent is selected from a small molecule, nucleotide, polynucleotide, protein, polypeptide, carbohydrate, lipid, and a combination thereof. In some embodiments, the agent comprises adipose tissue. In some embodiments, the adipose tissue comprises a lipoaspirate. In one embodiment, the drug delivery composition comprises about 10-90% by volume of lipoaspirate including all integers within the specified range. In another embodiment, the composition comprises about 25-90% by volume of lipoaspirate including all integers within the specified range. In some embodiments, the composition comprises at least 25% by volume of lipoaspirate. In other embodiments, the composition comprises at least 35% by volume of lipoaspirate. In other embodiments, the composition comprises at least 45% by volume of lipoaspirate. In yet other embodiments, the composition comprises at least 50% by volume of lipoaspirate. In other embodiments, the composition comprises at least 60% by volume of lipoaspirate. In other embodiments, the composition comprises at least 70% by volume of lipoaspirate. In other embodiments, the composition comprises at least 80% by volume of lipoaspirate. In other embodiments, the composition comprises at least 90% by volume of lipoaspirate.

In yet other embodiments, the Fractomers and or the drug delivery composition as detailed above can be formulated into a pharmaceutical composition in accordance with standard techniques well known to those skilled in the pharmaceutical art. Accordingly, a composition may comprise the Fractomer or aggregate thereof and/or a Fractomer or aggregate thereof and an effective amount of adipose tissue (e.g., lipoaspirate) along with one or more pharmaceutically acceptable carriers, excipients, or active pharmaceutical ingredients (APIs). The composition may be prepared for administration to a subject. Such compositions can be administered in dosages and by techniques well known to those skilled in the medical arts taking into consideration such factors as the age, sex, weight, and condition of the particular subject, and the route of administration.

The compositions can be administered prophylactically or therapeutically. In prophylactic administration, they can be administered in an amount sufficient to induce a response. In therapeutic applications, they are administered to a subject in need thereof in an amount sufficient to elicit a therapeutic effect. An amount adequate to accomplish this is defined as “therapeutically effective dose.” Amounts effective for this use will depend on, e.g., the particular composition of the Fractomer regimen administered, the manner of administration, the stage and severity of the disease, the general state of health of the patient, and the judgment of the prescribing physician, in some embodiments, the Fractomer may be co-administered with an agent, cells, adipose tissue (e.g., lipoaspirate) or a combination thereof.

The compositions provided herein can be administered by methods well known in the art as described in Donnelly et al., Ann. Rev. Immunol. 15: 617-648 (1997); FeLgner et al., U.S. Pat. No. 5,580,859; Feigner, U.S. Pat. No. 5,703,055; and Carson et al. U.S. Pat. No. 5,679,647, the contents of all of which are incorporated herein by reference in their entirety. The Fractomer can be complexed to particles or beads that can be administered to an individual, for example, using a vaccine gun. One skilled in the art would know that the choice of a pharmaceutically acceptable carrier, including a physiologically acceptable compound, depends, for example, on the route of administration.

The compositions can be delivered via a variety of routes. Typical delivery routes include parenteral administration, e.g., intradermal, intramuscular, or subcutaneous delivery. Other routes include oral administration, intranasal, intravaginal, transdermal, intravenous, intraarterial, intratumoral, intraperitoneal, and epidermal routes. In some embodiments, the Fractomer is administered intravenously, intraarterially, or intraperitoneally to the subject.

### Methods

The compositions provided herein can be used in numerous methods. One aspect of the present disclosure provides a method of augmenting autologous fat grafts in a subject, the method comprising, consisting of, or consisting essentially of administering to the subject a therapeutically effective amount of a composition as provided herein such that the autologous fat grafts are augmented in the subject.

Yet another aspect of the present disclosure provides a method of augmenting autologous fat grafts in a subject, the method comprising, comprising, consisting of, or consisting essentially of co-administering to the subject a therapeutically effective amount of a Fractomer as provided herein and a therapeutically effective amount of adipose tissue. In some embodiments, the adipose tissue comprises a lipoaspirate. In one embodiment, the Fractomer and adipose tissue are administered concurrently. In another embodiment, the Fractomer is administered prior to the adipose tissue. In another embodiment, the Fractomer is administered after the adipose tissue.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:
Clause 1. A tissue matrix composition comprising:
   a recombinant partially ordered polypeptide (Fractomer); and
   adipose tissue.
Clause 2. The composition of clause 1, wherein the Fractomer comprises:
   a plurality of disordered domains; and
   a plurality of structured domains.
Clause 3. The composition of clause 2, wherein
   the disordered domain comprises a plurality of an amino acid sequence of (GXGVP)ₙ (SEQ ID NO:1), wherein X is any amino acid except proline and n is an integer greater than or equal to 1; and
   the structured domain comprises a polyalanine domain.
Clause 4. The composition of clause 2, wherein the disordered domain comprises a plurality of an amino acid sequence of (GXGVP)ₙ (SEQ ID NO: 2), wherein X is Val (SEQ ID NO: 3), or Ala (SEQ ID NO: 4), or mixture of Ala and Val, and wherein n is an integer from 1 to 50.
Clause 5. The composition of clause 4, wherein X is an alternating iteration of Ala and Val in a ratio from 10:1 to 1:10 (Ala:Val).
Clause 6. The composition of clause 5, wherein X is an alternating iteration of Ala and Val in a ratio of 1:1 (SEQ ID NO: 5) or 1:4 (SEQ ID NO: 6)
Clause 7. The composition of clause 3, wherein the polyalanine domain comprises (Ala)ₘ, wherein m is an integer from 5 to 50.
Clause 8. The composition of clause 3, wherein the polyalanine domain comprises one or more of:

(SEQ ID NO: 7)
(A)₂₅;
(SEQ ID NO: 8)
K(A)₂₅K;
(SEQ ID NO: 9)
D(A)₂₅K;
(SEQ ID NO: 10)
GD(A₂₅)K;
or
(SEQ ID NO: 11)
GK(A₂₅)K.

Clause 9. The composition of clause 3, wherein the polypeptide comprises:

[(GXGVP)ₙ-GX¹(A)₂₅X¹]ₘ;

where X is A or V; X¹ is K or D; n is an integer from 10 to 20; and m is an integer from 4 to 8 ([(SEQ ID NO: 2)ₙ-(SEQ ID NO: 10 or 11)]ₘ).
Clause 10. The composition of clause 3 wherein the polypeptide comprises one or more of:

(SEQ ID NO: 12)
M[(GVGVP)₁₅-GD(A25)K]-GWP;
(SEQ ID NO: 13)
M[(GVGVP)₁₅-GD(A25)K]₄-GWP;
(SEQ ID NO: 14)
M[(GVGVP)₁₅-GK(A25)K]₆-GWP;
(SEQ ID NO: 15)
M[(GVGVP)₁₅-GK(A25)K]₄-GWP;
(SEQ ID NO: 16)
M[(G[A1:V1]GVP)₁₆-GD(A₂₅)K]₆-GWP;
(SEQ ID NO: 17)
M[(G[A1:V1]GVP)₁₆-GD(A₂₅)K]₄-GWP;
(SEQ ID NO: 18)
M[(G[V4:A1]GVP)₁₅-GD(A₂₅)K]₆-GWP;
or
(SEQ ID NO: 19)
M[(G[V4:A1]GVP)₁₅-GD(A₂₅)K]₄-GWP.

Clause 11. The composition of clause 3, wherein the polypeptide comprises one or more of:

(SEQ ID NO: 12)
M[(GVGVP)₁₅-GD(A₂₅)K]₆-GWP;
or
(SEQ ID NO: 18)
M[(G[V4:A1]GVP)₁₅-GD(A₂₅)K]₆-GWP.

Clause 12. The composition of any one of claims 1-11, wherein the Fractomer has a transition temperature of heating (T_{t-heating}) and a transition temperature of cooling (T_{t-cooling}).
Clause 13. The composition of clause 12, wherein the transition temperature of cooling (T_{t-cooling}) is concentration-independent.
Clause 14. The composition of clause 12, wherein the transition temperature of heating (T_{t-heating}) and the transition temperature of cooling (T_{t-cooling}) range from about 10° C. to about 45° C.
Clause 15. The composition of any one of claims 12-14, wherein the Fractomer forms a solid aggregate above the T_{t-heating}.
Clause 16. The composition of clause 15, wherein the solid aggregate resolubilizes when cooled to below the T_{t-cooling}.
Clause 17. The composition of clause 15, wherein the solid aggregate is a stable three-dimensional matrix.
Clause 18. The composition of clause 15, wherein the solid aggregate comprises a plurality of micropores.
Clause 19. The composition of clause 18, wherein the plurality of micropores range in size from about 1 μm to about 150 μm.
Clause 20. The composition of clause 1, wherein the composition comprises between about 200 μM and about 2 mM of Fractomer.
Clause 21. The composition of clause 1, wherein the adipose tissue comprises lipoaspirate.
Clause 22. The composition of clause 21, wherein the composition comprises a range of lipoaspirate from about 10% to about 90% by volume.
Clause 23. The composition of clause 21 or 22, wherein the composition comprises a range of lipoaspirate from about 25% to about 75% by volume.
Clause 24. The composition of any one of claims 22-23, wherein the composition comprises about 50% by volume lipoaspirate.
Clause 25. The composition of clause 21, wherein the composition comprises a mixture of Fractomer and lipoaspirate in a ratio ranging from about 1:9 to about 9:1.
Clause 26. The composition of clause 21, wherein the composition comprises a mixture of Fractomer and lipoaspirate in a ratio ranging from about 1:3 to about 3:1.
Clause 27. The composition of clause 21, wherein the composition comprises a mixture of Fractomer and lipoaspirate in a ratio of about 1:1.
Clause 28. The composition of clause 1, wherein the composition is a shapeable liquid or semisolid.
Clause 29. The composition of clause 1, wherein the composition is injectable or implantable.
Clause 30. The composition of clause 1, wherein the composition is shapeable or moldable into 2- or 3-dimensional shapes, areas, or volumes.
Clause 31. The composition of clause 1, wherein the Fractomer permits cell infiltration and vascularization of the adipose tissue.
Clause 32. A method of augmenting autologous fat grafts in a subject, the method comprising: administering to the subject a therapeutically effective amount of the composition of clause 1, such that the autologous fat grafts are augmented in the subject.
Clause 33. A method of augmenting an autologous fat graft in a subject, the method comprising:
   co-administering to the subject a therapeutically effective amount of a recombinant partially ordered polypeptide (Fractomer) and a therapeutically effective amount of adipose tissue.
Clause 34. The method of clause 33, wherein the adipose tissue comprises a lipoaspirate.
Clause 35. The method of clause 33, wherein the Fractomer and adipose tissue are administered concurrently or sequentially.
Clause 36. The method of clause 35, wherein the Fractomer and adipose tissue are administered sequentially, and the Fractomer is administered prior to the administration of the adipose tissue.
Clause 37. The method of clause 35, wherein the Fractomer and adipose tissue are administered sequentially, and the adipose tissue is administered prior to the administration of the Fractomer.
Clause 38. The method of clause 35, wherein the Fractomer and adipose tissue are combined in vitro, shaped or molded into 2- or 3-dimensional shapes, areas, or volumes, and implanted in situ in the subject.
Clause 39. The method of clause 33, wherein the Fractomer and adipose tissue are a shapeable liquid, semisolid, or molded semisolid prior to administration and following administration, form a solid aggregate.
Clause 40. The method of clause 39, wherein the Fractomer and adipose tissue are co-administered to a subject below the T_{t-heating} of the Fractomer and the Fractomer and adipose tissue form a solid after exposure to the subject's body temperature.
Clause 41. The method of clause 33, wherein the Fractomer permits cell infiltration and vascularization of the adipose tissue.
Clause 42. A method for preparing an autologous fat graft composition, the method comprising:
   (a) obtaining adipose tissue from a subject; and
   (b) combining a recombinant partially ordered polypeptide (Fractomer) with the adipose tissue of step (a) below the T_{t-heating} of the Fractomer to form a mixture.
Clause 43. The method of clause 44, further comprising:
   (c) shaping the mixture into shapes, areas, or volumes.
Clause 44. The method of clause 42 or 43, further comprising:
   (d) co-administering the mixture to the subject by injection or implantation.
Clause 45. The method of clause 44, wherein the mixture forms a solid aggregate at a temperature above the T_{t-heating} of the Fractomer.
Clause 46. A kit comprising a recombinant partially ordered polypeptide (Fractomer), and one or more of containers for combination, molds for specific volumetric dimensions, or a means for adipose tissue aspiration and/or administration.
Clause 47. Use of a therapeutically effective amount of a recombinant partially ordered polypeptide (Fractomer) and a therapeutically effective amount of adipose tissue for autologous fat grafting in a subject in need thereof.

### EXAMPLES

### Example 1

Method of Mixing Fractomer with Adipose Tissue (Fat)

Fat grafting is a clinical process of using liposuction to remove fat tissue from one part of a patient's body, and, during the same procedure, injecting that material into a new area for reconstruction or cosmetic enhancement. Liposuctioned fat is processed to remove excess blood, oil, and non-fat tissue, and the processed tissue (called lipoaspirate) is collected in a syringe for injection back into the patient (in another body area).

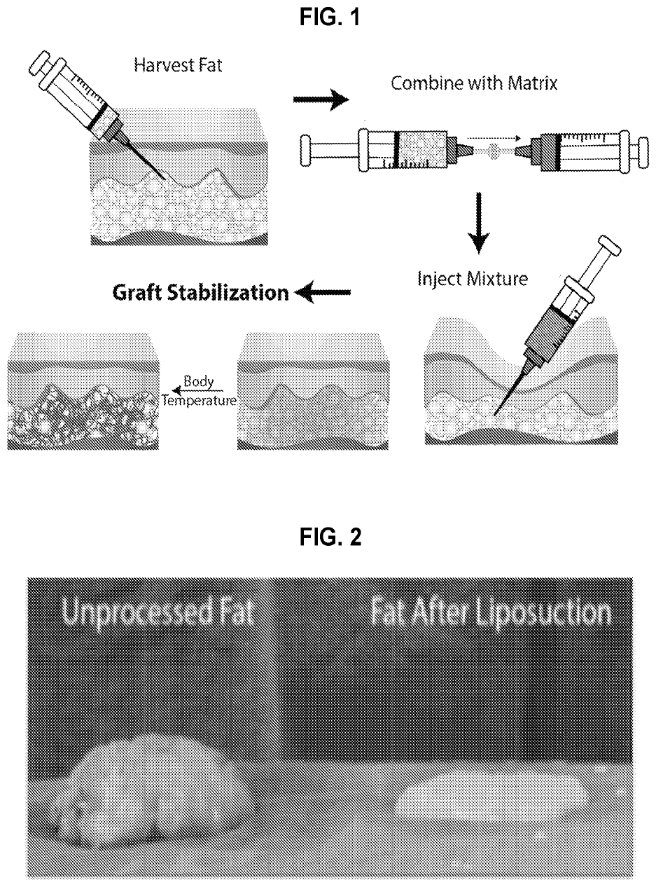

Purified lipoaspirate can be combined with liquid Fractomer for injection into the area being reconstructed. FIG. 1 shows a proposed method of combining Fractomer with harvested fat for re-injection of stabilized fat grafts. Harvested lipoaspirate is mixed with Fractomer matrix solution and the lipoaspirate:Fractomer mixture is then injected into a deficient area of a patient. Increased body temperature causes the Fractomer to form a solid scaffold structure, which leads to graft stabilization. Purified lipoaspirate can also be combined with liquid Fractomer in a heated ex vivo mold, where the Fractomer aggregates when heated, stabilizing the molded fat shape, which can then be implanted into the desired area of a patient.

FIG. 2 illustrates the structural differences between unprocessed fat and processed lipoaspirate. After liposuction and processing, fat loses its solid-like properties and does not have the mechanical integrity to maintain shape or volume. By mixing lipoaspirate with Fractomer, structural and mechanical integrity can be reinforced to help maintain shape and volume retention of fat grafts.

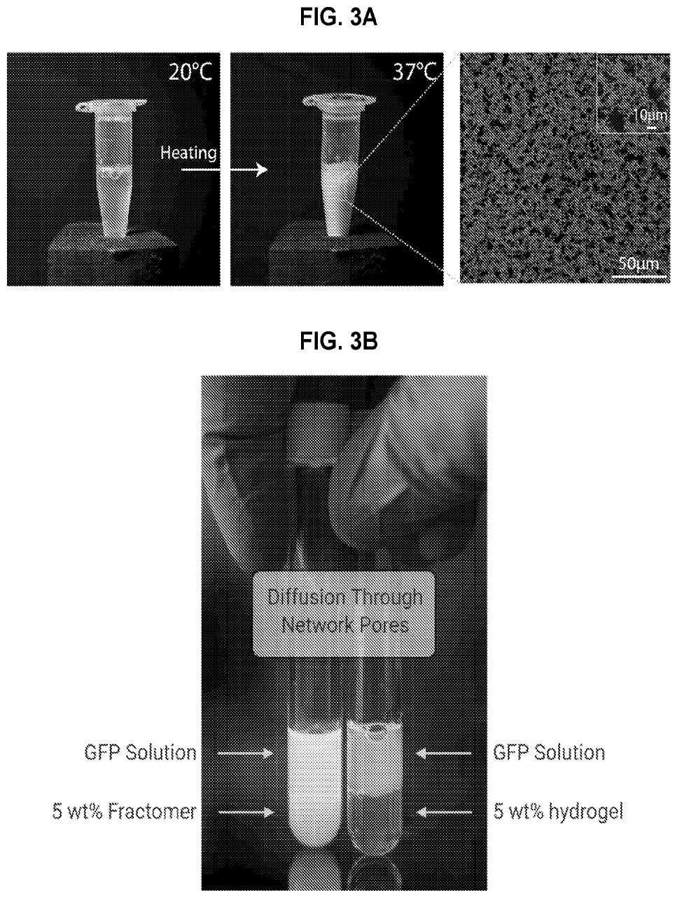

Fractomers (SEQ ID NO: 12) are thermally responsive and form a solid, porous elastin-like network when heated (FIG. 3A). Fractomers are liquid and injectable at room temperature but aggregate and form a solid, porous network at body temperature, where the higher temperature initiates Fractomer scaffold formation with entrapped lipoaspirate. The threshold temperature at which Fractomer undergoes phase separation into a solid, porous network is tunable. By tuning the composition and segment organization of Fractomers, it is possible to design mechanically stable networks with a high surface to volume ratio and a fractal dimension similar to native elastin. The porous network of Fractomer (SEQ ID NO: 12) at higher temperatures also allows greater nutrient flow compared to traditional hydrogels (FIG. 3B).

### Example 2

### Fractomers are Shelf-Stable in Several Storage Conditions

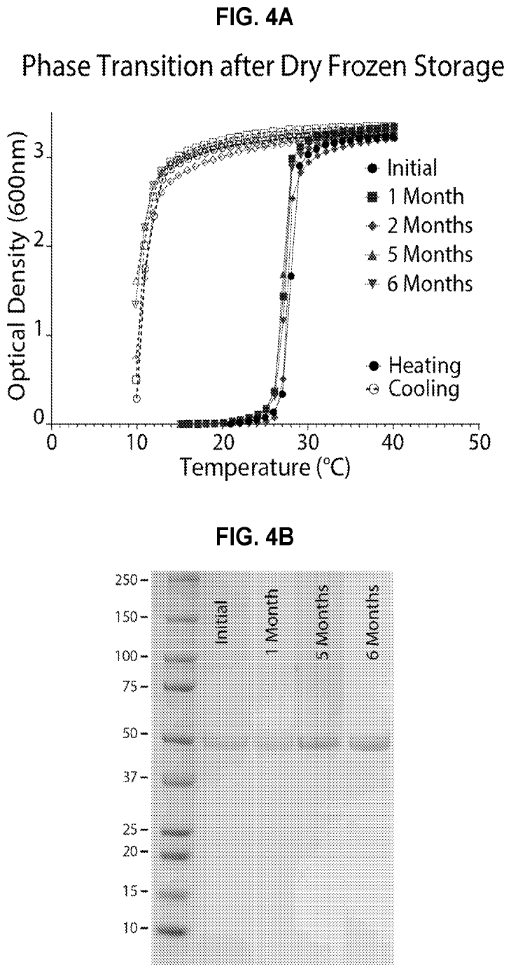

To evaluate the shelf life of Fractomers, at least 3 batches (to ensure replication) of high-purity Fractomer (SEQ ID NO: 12) were parsed into aliquots and stored under three different conditions: (1) lyophilized and stored at room temperature, (2) lyophilized and stored at −20° C., (3) resuspended in PBS and stored at −20° C. At periodic time points out to 6 months, the incidence of protein degradation was evaluated using UV spectrophotometry optical density and SDS-PAGE (FIG. 4A-B). Because Fractomer proteins are highly disordered, and therefore functionally denatured at all times, they have significantly reduced storage requirements compared to most proteins. Fractomer samples that were lyophilized and stored at −20° C. showed no shift in the precise T_{t-heating} or T_{t-cooling} phase transition overtime (FIG. 4A), and samples stored under the other two conditions showed similar results. Additionally, no loss or shift in the target Fractomer protein band was observed by SDS-PAGE at any time point, and no truncation/degradation products, impurities, or other additional bands were observed (FIG. 4B).

### Example 3

### Fractomers can Encapsulate Healthy Adipocytes and Improve Shape and Projection

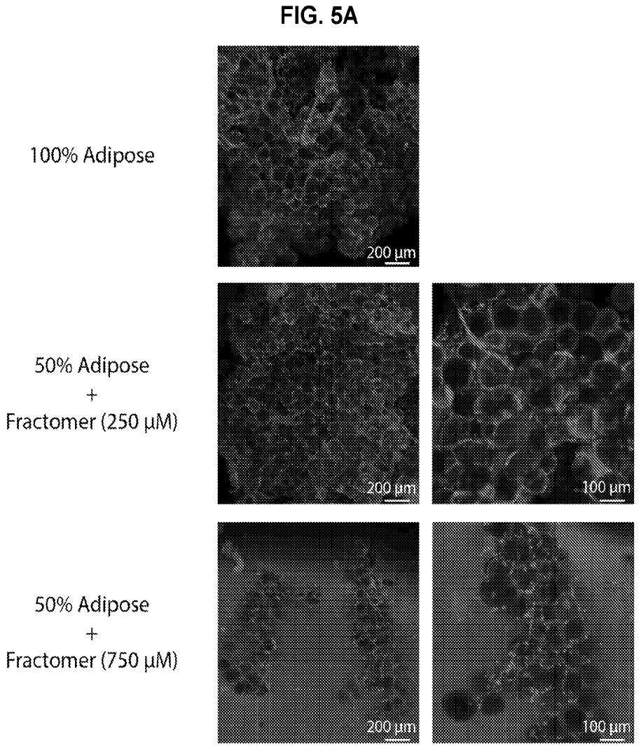

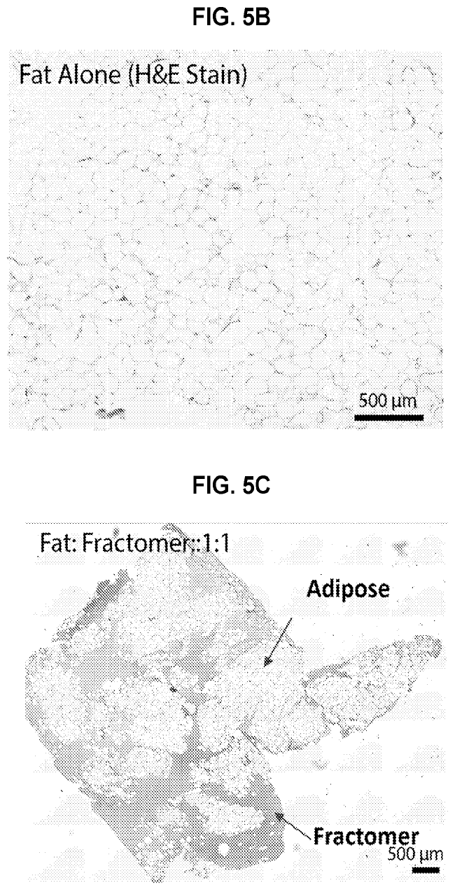

Discarded human lipoaspirate was mixed with different Fractomer concentrations and analyzed using confocal microscopy and histology (FIG. 5A-C). In all cases, the lipoaspirate was composed of >90% large adipocytes clumped into groups of 50-500 cells, which are fragments small enough to pass through a liposuction cannula. Fractomer concentrations ranging from 250 μM to 1 mM were mixed in a 1:1 ratio with the lipoaspirate at room temperature, and then placed in a 37° C. incubator for 30 min before either (a) staining and imaging using confocal microscopy, or (b) overnight fixation in 10% formalin followed by paraffin embedding and H & E staining for histological analysis.

FIG. 5A shows confocal microscopy analysis of lipoaspirate:Fractomer (SEQ ID NO: 12) mixtures. Lipoaspirate was mixed with different Fractomer concentrations (low=250 μM, high=750 μM) in a ratio of 1:1 and stained with LipidTOX (red, also stains Fractomer) and DAPI (blue). Imaging revealed no changes in adipocyte structure or viability. Although Fractomer was observed in the small gaps between individual adipocytes, Fractomer primarily functions as a type of highly porous “glue” holding clumps of adipocytes together.

FIG. 5B-C show histological analysis of fat alone compared to a 1:1 ratio mixture of lipoaspirate:Fractomer (SEQ ID NO: 18). Lipoaspirate alone and lipoaspirate mixed with Fractomer (250 μM) were fixed and stained with H & E. Fractomer can be seen interspersed between areas of more concentrated fat cells.

### Example 4

### Creating 3D Molds Using Fractomer Alone and Fat+Fractomer

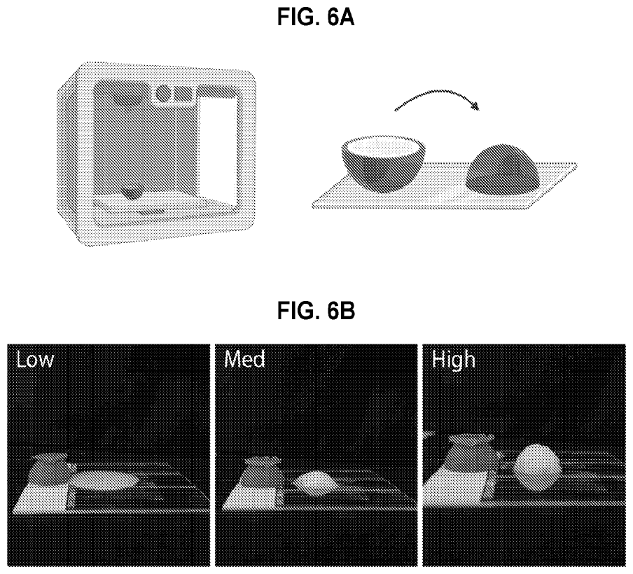

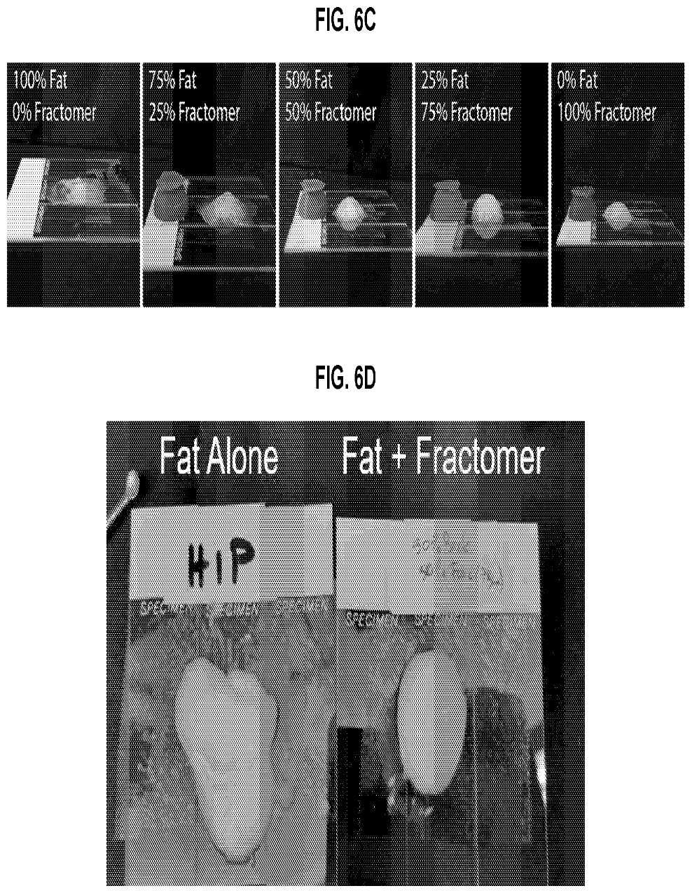

The ability of Fractomer to maintain shape and projection when co-injected with fat was also evaluated using 3D printed hemispherical molds. Fractomer (SEQ ID NO: 12) was injected into the molds in a liquid state and allowed to aggregate at 37° C. for 5 min before removal from the molds (FIG. 6A). When using Fractomer alone (SEQ ID NO: 12) in concentrations ranging from 200 μM to 2 mM, projection maintenance scaled with a minimum concentration of ˜500 μM was needed to preserve shape (FIG. 6B). FIG. 6B demonstrates that molding Fractomer alone at low (500 μM), medium (750 μM), and high (2 mM) concentrations shows increased shape preservation with increasing concentration. Fractomer solution (750 μM) (SEQ ID NO: 12) was further mixed with human lipoaspirate at various ratios to determine its effect on fat shape stability (FIG. 6C). Fractomer was able to significantly improve the shape preservation of lipoaspirate which, alone, simply behaves as an oily liquid (FIG. 6C). Equivalent results were observed when Fractomer solution (750 μM) (SEQ ID NO: 18) was mixed in a 1:1 ratio with liposuctioned porcine fat in a 3D printed mold and placed at 37° C. to allow the Fractomer to aggregate (FIG. 6D). These results suggest surprising and unexpected complementary mechanical interactions when fat is mixed with Fractomer.

Overall, mixtures of lipoaspirate with Fractomer were found to improve shape retention after release from the molds. The most surprising and unexpected finding was the shapeability of the fat+Fractomer mixtures. Even after forming, the encapsulated fat could be molded, shaped, cut and generally manipulated without loss of structural integrity. The ability to manipulate this material in vivo to the desired shape provides an unprecedented amount of control to surgeons using lipoaspirate to fill and rebuild defects in patients.

### Example 5

### Shape of Fractomer can be Controlled on Injection

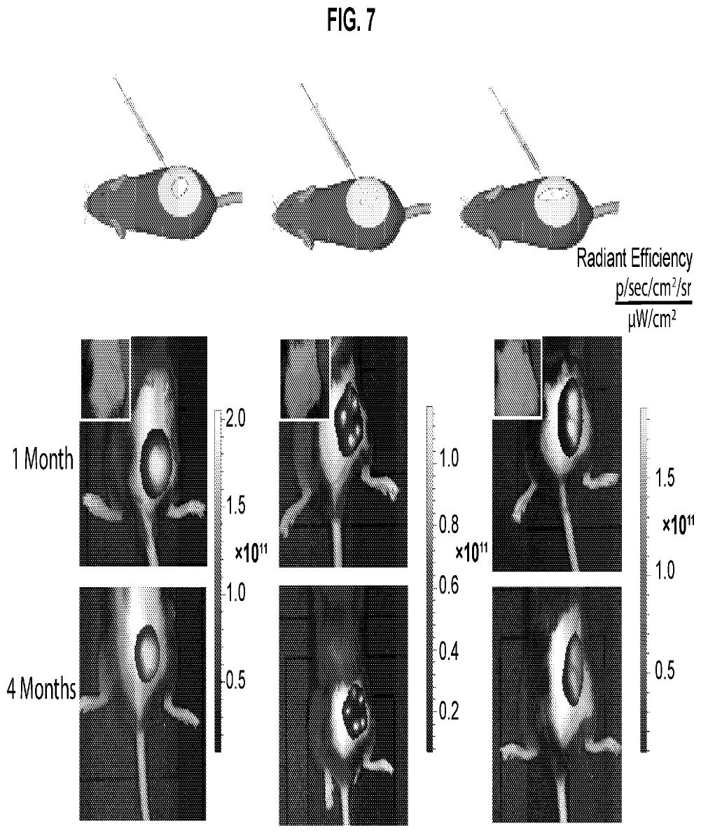

The shape of Fractomer injections can also be controlled based on the pattern of injection. Fluorescently labeled Fractomer solution (750 μM) (SEQ ID NO: 12) was injected into the hind flank of mice in three different shape patterns, and the shapes were monitored at 1 month and 4 months post-injection using IVIS Spectrum imaging. For all three shapes-sphere, elongated rod, and dispersed dots—the injections maintained their desired shape throughout the lifetime of the injection (FIG. 7).

### Example 6

Fractomer Shows Long-Term Stability and that Degradation is Controllable with Concentration

When used to augment fat grafting, Fractomer should be designed to degrade and resorb on a timeline that aligns with fat proliferation, allowing fat cells to replace Fractomer volume as it is being broken down into non-toxic amino acids. Controlled degradation, ultimately resulting in tissue grafts composed entirely of the patients' own tissue, avoids long term risk of foreign body response since Fractomer is not intended to be a permanent implant.

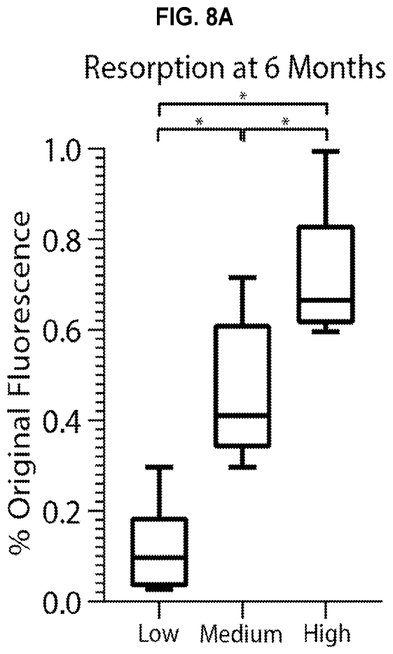

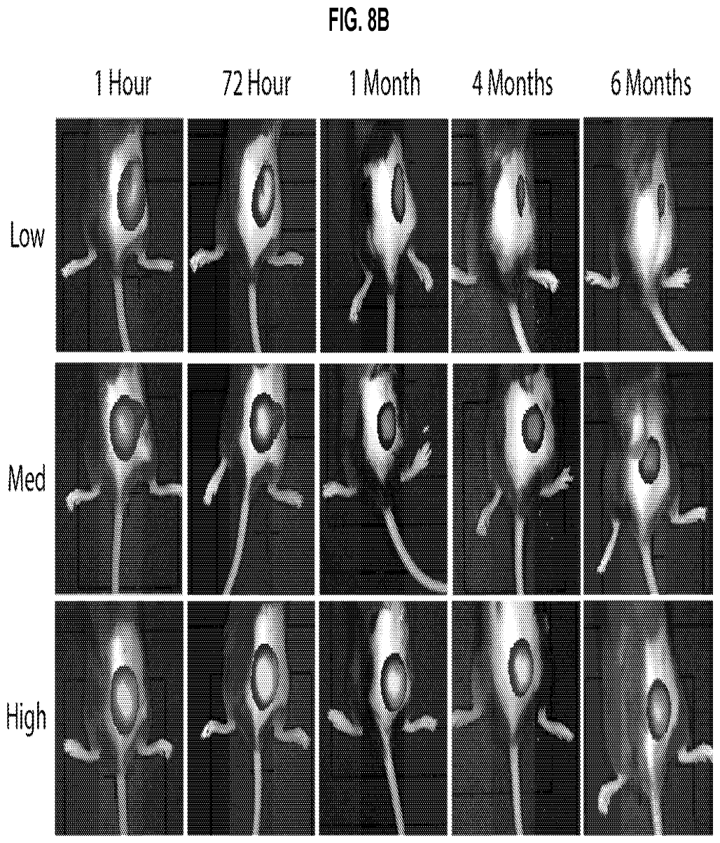

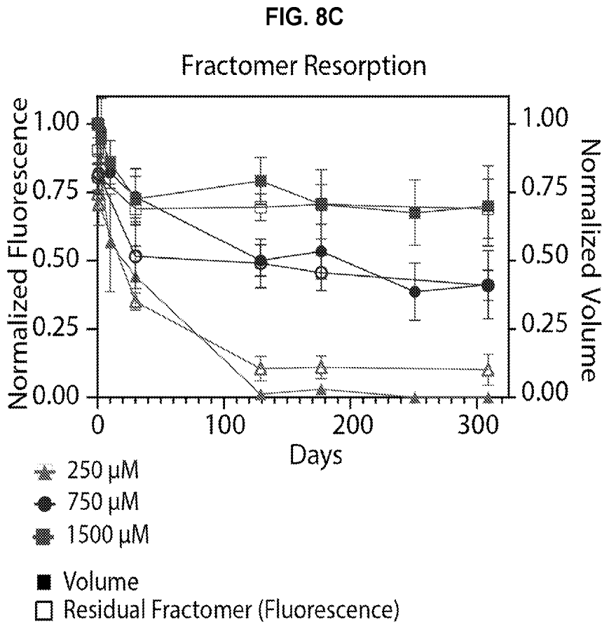

To evaluate the long-term stability of Fractomer, different formulations of Fractomer (SEQ ID NO: 12) were fluorescently labeled with a near infrared dye and injected into BL/6 immunocompetent mice. The use of BL/6 mice ensured that immune-regulated degradation would be captured if present. The rate of degradation and the in vivo stability of the Fractomer protein was subsequently tracked over 10 months using IVIS Spectrum in vivo imaging. A snapshot of fluorescence retention at 6 months demonstrates that Fractomer degradation can be controlled with concentration (low=250 μM, med=750 μM, high=1500 μM), as fluorescence intensity was significantly higher for the medium and high concentrations of Fractomer compared to the low concentration (FIG. 8A). Representative fluorescence spectroscopy images in BL/6 mice injected with the different Fractomer concentration formulations are shown in FIG. 8B.

The use of a low concentration formulation of Fractomer (250 μM) continued to be resorbed out to 3 months (FIG. 8C). While the presence of Fractomer was evident in the fluorescence scan, no visible or palpable volume was evident (FIG. 8C). Medium (750 μM) and high (1500 μM) concentration formulations of Fractomer were found to be more stable with longer in vivo persistence (FIG. 8C).

### Example 7

### Fractomer Improves Fat Survivability and Volume Retention

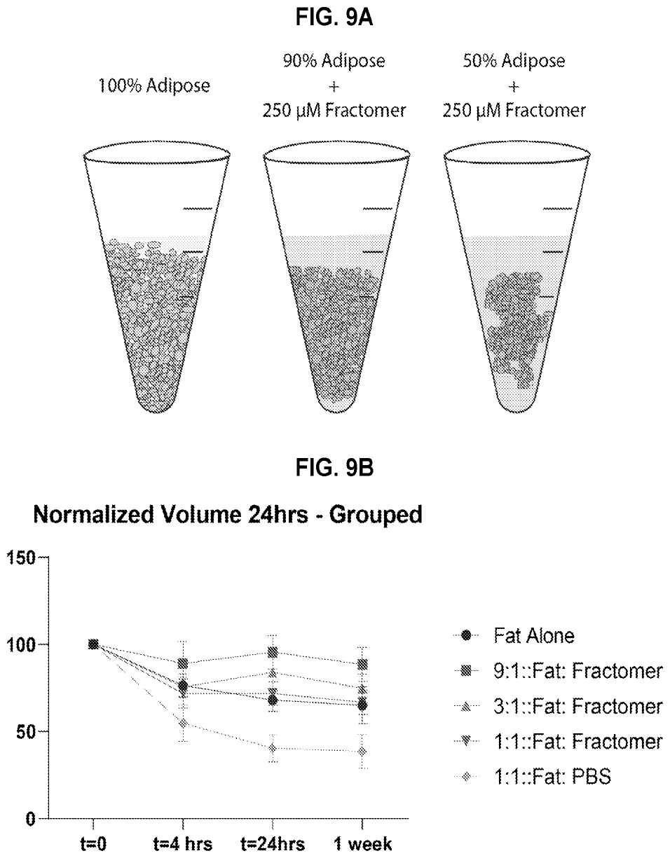

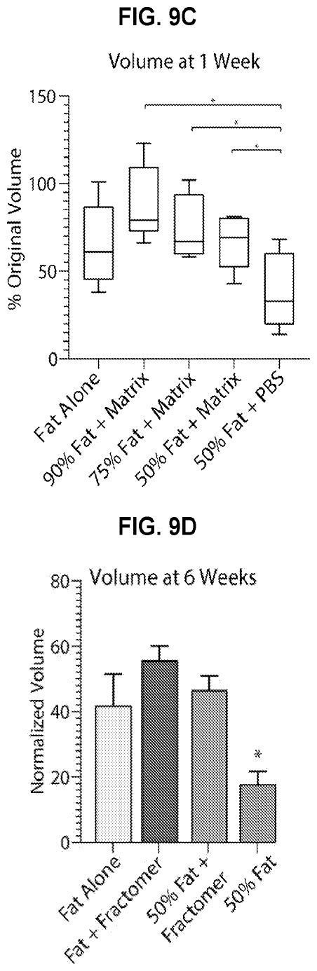

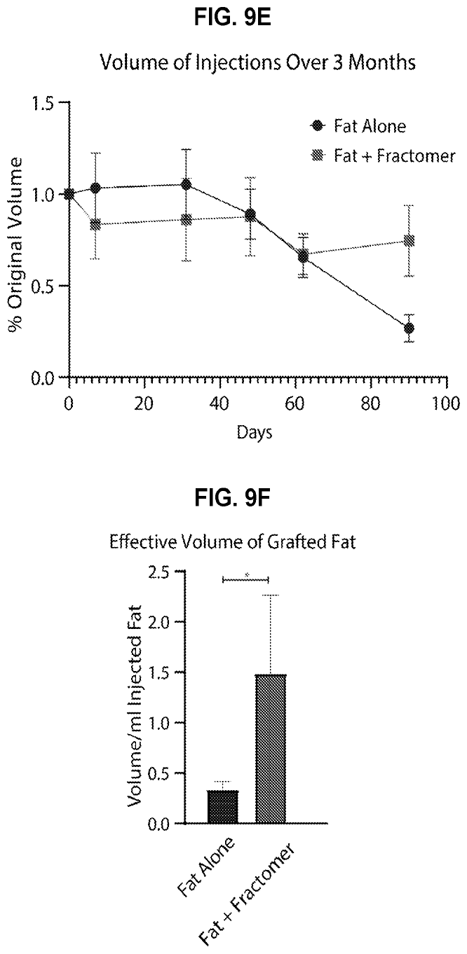

Using a low concentration (250 μM) solution of Fractomer (SEQ ID NO: 12), fat:Fractomer ratios were first screened to determine the effect of reducing fat in the grafts. Injection mixtures of fat alone (100% adipose), 90% by volume of fat+Fractomer solution, 75% by volume of fat+Fractomer solution, 50% by volume of fat+Fractomer solution r, and a 50% by volume of fat+PBS control were evaluated over 6 weeks in the nu/nu mouse strain. Nu/nu mice were selected to prevent rejection of human adipose tissue. Example graphics of the different fat:Fractomer solution (250 μM) mixtures used in experimental groups for injection into nu/nu mice are shown in FIG. 9A.

Following injection, the greatest volume loss was observed in the first 24 hr for the 50% by volume of fat+PBS control group (FIG. 9B). This was likely due to the presence of residual oils in the fat mixtures that were not removed during the fat purification process in the operating room, which is common in the clinic. The fat grafts showed improvements in volume retention over 1 week when mixed with the different ratios of Fractomer matrix (FIG. 9B-C). At 6 weeks post-injection, at least 50% less adipose tissue could be used to achieve the same final volume as fat alone (FIG. 9D). Volume retention was significantly lower when a reduced fat amount (50% by volume of fat) was used without the inclusion of the stabilizing Fractomer matrix (FIG. 9D).

To further examine fat volume retention improvements, a higher concentration (750 μM) of Fractomer was used and the observation time was extended (FIG. 9E). Injections of 50% by volume of fat+Fractomer solution were compared to fat alone injections over 3 months, at which time the experiment was halted due to harmful cyst formations in the fat alone control group. Use of this higher concentration formulation of Fractomer showed a higher volume retention (FIG. 9E). Normalized to the amount of initial fat injected, the effective volume of grafted fat at 3 months was tripled by combining the fat with Fractomer (FIG. 9F).

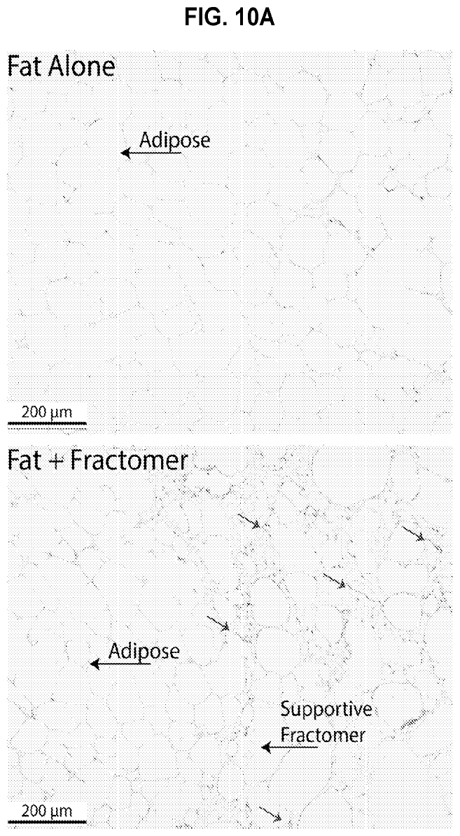

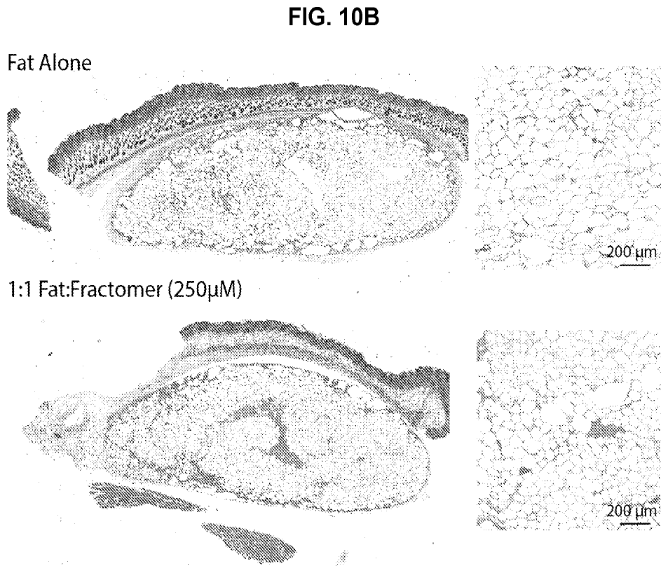

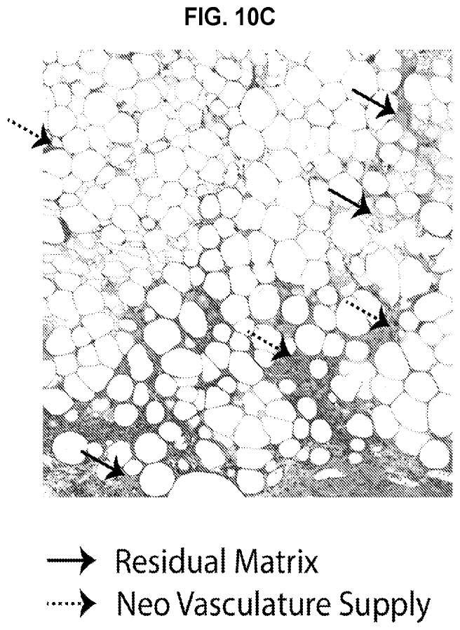

Histological analysis of the implants revealed that the vasculature supply is high within fat grafts made with Fractomer (250 μM) compared to grafts made of fat alone (FIG. 10A). A high prevalence of CD31+ vasculature markers were observed in the supportive Fractomer sections between groups of adipocytes (FIG. 10A, small red arrows). The CD31+ staining reveals a high prevalence of blood vessels forming in both the surviving adipocyte groups and the Fractomer-heavy sections of the graft, suggesting that Fractomer is likely able to improve the survival of the implanted fat.

A histological comparison of the fat distribution at 1-month post-injection between fat alone and 1:1 fat:Fractomer (250 μM) mixtures indicated no differences in the graft cell compositions despite 50% by volume less fat being used in the fat+Fractomer experimental group (FIG. 10B). This indicates good incorporation of the fat:Fractomer graft and suggests that fat proliferation is likely occurring at a high level within the Fractomer matrix. Furthermore, evidence of residual Fractomer is observed at 30 days post-injection with no foreign body response, which would be indicative of chronic inflammation (FIG. 10C). Similar to histological results from in vitro mixtures, Fractomer appears to persist as a type of protein “glue,” maintaining volume between adipocyte groups while new ECM, cell infiltrates, and vascularization are given time to form.

### Example 8

### Cyst Formation and Necrosis in Fat Alone Injections is not Observed in Fat+Fractomer Mixtures

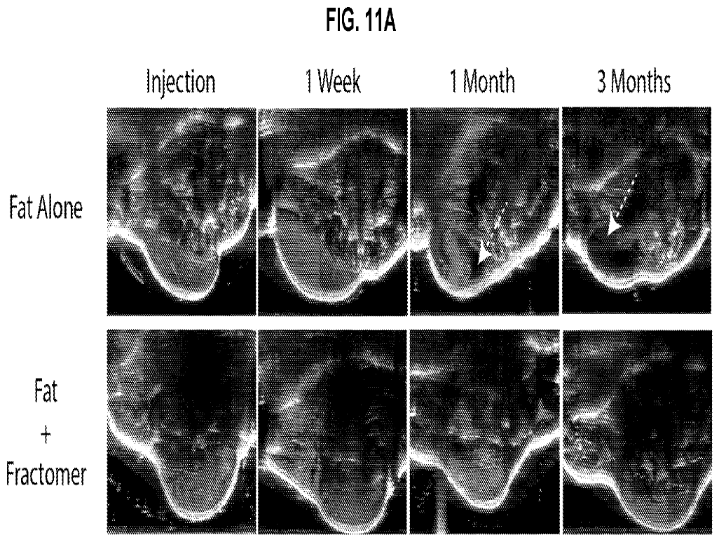

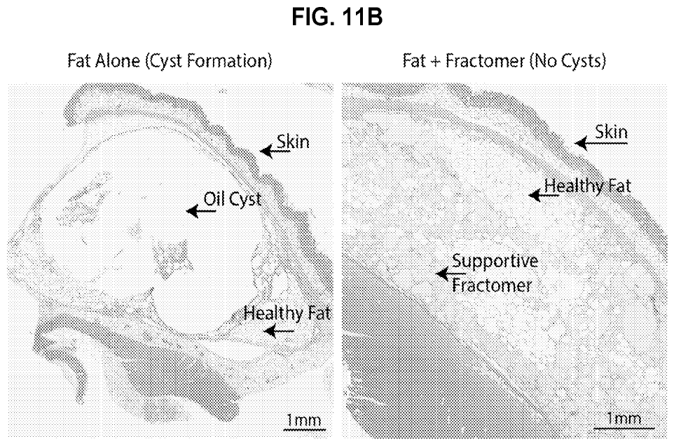

Although increasing the effective volume of fat tissue is a key metric for success when using Fractomer, the exact biological composition of that increased fat volume is equally important. For example, the avoidance of cyst formation, calcification, and necrosis is critical to the development of healthy fat tissue. Evidence of these anomalies in healthy fat formation was screened for by using a combination of ultrasound, histology, and micro-CT. Ultrasounds of fat grafts were captured over 3 months in mice injected with fat alone or a 1:1 mixture of lipoaspirate and Fractomer solution (750 μM) (SEQ ID NO: 12) (FIG. 11A). The formation of large oil cysts, which are a key marker of fat necrosis, was observed in the grafts of the fat alone group at 1 month and 3 months post-injection (FIG. 11A, top row panels, white arrows), while no cyst formation was observed for any mice injected with fat+Fractomer solution (FIG. 11A, bottom row panels). These cysts were observed by 1 month in >50% of the mice injected with fat alone. Additionally, these cysts were found to expand in volume throughout the time-course of 3 months, and in several mice, the cysts dominated the volume of the entire injection. Furthermore, a histological comparison of the cell compositions of fat alone and 1:1 fat:Fractomer solution (750 μM) grafts at 3 months shows stark evidence of large oil cyst formation in the fat alone group (FIG. 11B, left panel). However, no cyst formation was observed in any Fractomer grafts by histological analysis (FIG. 11b, right panel). Notably, the higher concentration of Fractomer solution (750 μM) led to higher retention of Fractomer at 3 months, and Fractomer can be seen supporting adipocytes. These results are consistent with data from the resorption studies using higher concentrations of Fractomer.

The formation of cysts in the fat alone group is consistent with clinical observation of fat necrosis in humans. Injections of 50% by volume of fat+Fractomer solution are heterogeneous mixtures of adipocytes and residual Fractomer, with clear signs of vascularization under contrast CT (not seen in the fat alone). Furthermore, there were no calcifications observed in the fat+Fractomer grafts, which would appear as punctate white markings in the mice. The improved health of the grafts with Fractomer can likely be attributed to the reduced amount of fat tissue used, which requires less initial metabolic demand. Additionally, the porous nature and rapid vascularization of the Fractomer can quickly provide nutrients for adipocyte survival and growth.

### Example 9

### Fractomer in a Porcine Model of Autologous Fat Grafting

Rodent models are sufficient for optimization of key variables, but small animals possess little subcutaneous fat tissue, and their soft tissue structure differs anatomically from humans. In addition, though the nude mouse is immunodeficient and can tolerate human cells, the use of human fat represents an important variation from autologous grafting, in both body response and available volume of injections. Therefore, long-term Fractomer enhanced fat grafting in a minipig model will be further evaluated. This proposed experiment will demonstrate that Fractomers can expand volume and create healthy fat tissue at clinically relevant volumes in an accepted porcine model of autologous fat grafting.

Minipigs will be anesthetized and a small midline incision will be created high on the back 3-4 cm adjacent to the spine. The subcutaneous adipose depot will be aspirated using power assisted liposuction with a 4 mm cannula. The goal is to harvest approximately 50 cm³ of adipose tissue. If necessary, an additional incision will be created to permit liposuction near the hind flank. The fat will be washed and processed using the Revovle™ system used for preparing human fat for grafting. Lipoaspirate will then be divided into four different injection formulation mixtures of ˜25 cm³ volume each, which will likely be used clinically for surgical revisions for breast reconstruction shape and small lumpectomies: 100% by volume of lipoaspirate (fat alone), 50% by volume of lipoaspirate+Fractomer solution, 25% by volume of lipoaspirate+Fractomer solution, or Fractomer alone. Each 25 cm³ mixture will be injected into a unique site in mammary fat. These groups will be duplicated with ˜3 mL injections along the flank of the pigs to mimic the use of Fractomer in non-breast applications. A critical component of this task will be an “ease-of-use” analysis of the Fractomer in a clinic-mimicking environment, allowing insight into the type of final product delivery techniques that ensure consistency with existing surgical practice. Pigs are then recovered, housed, and monitored.

The goal is to determine the volume retention of fat grafts stabilized with Fractomer and the cellular health/composition of that volume. Clinical fat volume retentions for autologous fat grafts are reported to be between 30-60% at 6 months, depending on the technique of implantation, and similar retention has been seen in minipig models. The volume of the fat combined with Fractomer will be maintained long term through sustained viability of initial injectate as well as proliferation or hypertrophy of the injected fat cells. This is a rational expectation because the consumption of oxygen and other nutrients per unit volume of injectate will be lower than pure fat when the fat is diluted into Fractomer matrix. The lower consumption per unit volume would increase the likelihood that diffusion would adequately deliver the required nutritional sustenance for the injected fat cells. These outcomes are being evaluated using a combination of 3D ultrasound and periodic biopsy. Imaging will permit the determination of the likelihood of cyst formation with the grafts. At days 10, 60, and 180, tissue will be extracted using needle biopsies (or full excision on day 180) for histological investigation of cell composition, fibrosis, neovascularization, necrosis, and chronic inflammation.

What is claimed:

1. A tissue matrix composition comprising:
a recombinant partially ordered polypeptide (Fractomer); and
adipose tissue.

2. The composition of claim 1, wherein the Fractomer comprises:
a plurality of disordered domains; and
a plurality of structured domains.

3. The composition of claim 2, wherein
the disordered domain comprises a plurality of an amino acid sequence of (GXGVP)ₙ (SEQ ID NO:1), wherein X is any amino acid except proline and n is an integer greater than or equal to 1; and
the structured domain comprises a polyalanine domain.

4. The composition of claim 2, wherein the disordered domain comprises a plurality of an amino acid sequence of (GXGVP)ₙ (SEQ ID NO: 2), wherein X is Val (SEQ ID NO: 3), or Ala (SEQ ID NO: 4), or mixture of Ala and Val, and wherein n is an integer from 1 to 50.

5. The composition of claim 4, wherein X is an alternating iteration of Ala and Val in a ratio from 10:1 to 1:10 (Ala:Val).

6. The composition of claim 5, wherein X is an alternating iteration of Ala and Val in a ratio of 1:1 (SEQ ID NO: 5) or 1:4 (SEQ ID NO: 6)

7. The composition of claim 3, wherein the polyalanine domain comprises (Ala)ₘ, wherein m is an integer from 5 to 50.

8. The composition of claim 3, wherein the polyalanine domain comprises one or more of:
(SEQ ID NO: 7)
(A)₂₅;
(SEQ ID NO: 8)
K(A)₂₅K;
(SEQ ID NO: 9)
D(A)₂₅K;
(SEQ ID NO: 10)
GD(A₂₅)K;
or
(SEQ ID NO: 11)
GK(A₂₅)K.

9. The composition of claim 3, wherein the polypeptide comprises:
[(GXGVP)ₙ-GX¹(A)₂₅X¹]ₘ;
where X is A or V; X¹ is K or D; n is an integer from 10 to 20; and m is an integer from 4 to 8 ([(SEQ ID NO: 2)ₙ-(SEQ ID NO: 10 or 11)]ₘ).

10. The composition of claim 3, wherein the polypeptide comprises one or more of:
(SEQ ID NO: 12)
M[(GVGVP)₁₅-GD(A25)K]₆-GWP;
(SEQ ID NO: 13)
M[(GVGVP)₁₅-GD(A25)K]₄-GWP;
(SEQ ID NO: 14)
M[(GVGVP)₁₅-GK(A25)K]₆-GWP;
(SEQ ID NO: 15)
M[(GVGVP)₁₅-GK(A25)K]₄-GWP;
(SEQ ID NO: 16)
M[(G[A1:V1]GVP)₁₆-GD(A₂₅)K]₆-GWP;
(SEQ ID NO: 17)
M[(G[A1:V1]GVP)₁₆-GD(A₂₅)K]₄-GWP;
(SEQ ID NO: 18)
M[(G[V4:A1]GVP)₁₅-GD(A25)K]₆-GWP;
or
(SEQ ID NO: 19)
M[(G[V4:A1]GVP)₁₅-GD(A₂₅)K]₄-GWP.

11. The composition of claim 3, wherein the polypeptide comprises one or more of:
(SEQ ID NO: 12)
M[(GVGVP)₁₅-GD(A₂₅)K]₆-GWP;
or
(SEQ ID NO: 18)
M[(G[V4:A1]GVP)₁₅-GD(A₂₅)K]₆-GWP

12. The composition of any one of claims 1-11, wherein the Fractomer has a transition temperature of heating (T_{t-heating}) and a transition temperature of cooling (T_{t-cooling}).

13. The composition of claim 12, wherein the transition temperature of cooling (T_{t-cooling}) is concentration-independent.

14. The composition of claim 12, wherein the transition temperature of heating (T_{t-heating}) and the transition temperature of cooling (T_{t-cooling}) range from about 10° C. to about 45° C.

15. The composition of any one of claims 12-14, wherein the Fractomer forms a solid aggregate above the T_{t-heating}.

16. The composition of claim 15, wherein the solid aggregate resolubilizes when cooled to below the T_{t-cooling}.

17. The composition of claim 15, wherein the solid aggregate is a stable three-dimensional matrix.

18. The composition of claim 15, wherein the solid aggregate comprises a plurality of micropores.

19. The composition of claim 18, wherein the plurality of micropores range in size from about 1 μm to about 150 μm.

20. The composition of claim 1, wherein the composition comprises between about 200 μM and about 2 mM of Fractomer.

21. The composition of claim 1, wherein the adipose tissue comprises lipoaspirate.

22. The composition of claim 21, wherein the composition comprises a range of lipoaspirate from about 10% to about 90% by volume.

23. The composition of claim 21 or 22, wherein the composition comprises a range of lipoaspirate from about 25% to about 75% by volume.

24. The composition of any one of claims 22-23, wherein the composition comprises about 50% by volume lipoaspirate.

25. The composition of claim 21, wherein the composition comprises a mixture of Fractomer and lipoaspirate in a ratio ranging from about 1:9 to about 9:1.

26. The composition of claim 21, wherein the composition comprises a mixture of Fractomer and lipoaspirate in a ratio ranging from about 1:3 to about 3:1.

27. The composition of claim 21, wherein the composition comprises a mixture of Fractomer and lipoaspirate in a ratio of about 1:1.

28. The composition of claim 1, wherein the composition is a shapeable liquid or semisolid.

29. The composition of claim 1, wherein the composition is injectable or implantable.

30. The composition of claim 1, wherein the composition is shapeable or moldable into 2- or 3-dimensional shapes, areas, or volumes.

31. The composition of claim 1, wherein the Fractomer permits cell infiltration and vascularization of the adipose tissue.

32. A method of augmenting autologous fat grafts in a subject, the method comprising:
administering to the subject a therapeutically effective amount of the composition of claim 1, such that the autologous fat grafts are augmented in the subject.

33. A method of augmenting an autologous fat graft in a subject, the method comprising:
co-administering to the subject a therapeutically effective amount of a recombinant partially ordered polypeptide (Fractomer) and a therapeutically effective amount of adipose tissue.

34. The method of claim 33, wherein the adipose tissue comprises a lipoaspirate.

35. The method of claim 33, wherein the Fractomer and adipose tissue are administered concurrently or sequentially.

36. The method of claim 35, wherein the Fractomer and adipose tissue are administered sequentially, and the Fractomer is administered prior to the administration of the adipose tissue.

37. The method of claim 35, wherein the Fractomer and adipose tissue are administered sequentially, and the adipose tissue is administered prior to the administration of the Fractomer.

38. The method of claim 35, wherein the Fractomer and adipose tissue are combined in vitro, shaped or molded into 2- or 3-dimensional shapes, areas, or volumes, and implanted in situ in the subject.

39. The method of claim 33, wherein the Fractomer and adipose tissue are a shapeable liquid, semisolid, or molded semisolid prior to administration and following administration, form a solid aggregate.

40. The method of claim 39, wherein the Fractomer and adipose tissue are co-administered to a subject below the T_{t-heating} of the Fractomer and the Fractomer and adipose tissue form a solid after exposure to the subject's body temperature.

41. The method of claim 33, wherein the Fractomer permits cell infiltration and vascularization of the adipose tissue.

42. A method for preparing an autologous fat graft composition, the method comprising:
(a) obtaining adipose tissue from a subject; and
(b) combining a recombinant partially ordered polypeptide (Fractomer) with the adipose tissue of step (a) below the T_{t-heating} of the Fractomer to form a mixture.

43. The method of claim 44, further comprising:
(c) shaping the mixture into shapes, areas, or volumes.

44. The method of claim 42 or 43, further comprising:
(d) co-administering the mixture to the subject by injection or implantation.

45. The method of claim 44, wherein the mixture forms a solid aggregate at a temperature above the T_{t-heating} of the Fractomer.

46. A kit comprising a recombinant partially ordered polypeptide (Fractomer), and one or more of containers for combination, molds for specific volumetric dimensions, or a means for adipose tissue aspiration and/or administration.

47. Use of a therapeutically effective amount of a recombinant partially ordered polypeptide (Fractomer) and a therapeutically effective amount of adipose tissue for autologous fat grafting in a subject in need thereof.


SEQUENCE LISTING (22 sequences)

SEQ ID NO: 1 - protein, 5 aa, Artificial Sequence
  Synthetic
  MISC_FEATURE (1)..(5): Sequence can be repeated 1 or more times
  MISC_FEATURE (2)..(2): Xaa is any amino acid except proline
GXGVP

SEQ ID NO: 2 - protein, 5 aa, Artificial Sequence
  Synthetic
  MISC_FEATURE (1)..(5): Sequence can be repeated 1 or more times
  MISC_FEATURE (2)..(2): Xaa is Ala or Val
GXGVP

SEQ ID NO: 3 - protein, 5 aa, Artificial Sequence
  Synthetic
  MISC_FEATURE (1)..(5): Sequence can be repeated 1 or more times
GAGVP

SEQ ID NO: 4 - protein, 5 aa, Artificial Sequence
  Synthetic
  MISC_FEATURE (1)..(5): Sequence can be repeated 1 or more times
GVGVP

SEQ ID NO: 5 - protein, 10 aa, Artificial Sequence
  Synthetic
  MISC_FEATURE (1)..(10): G[A1V1]GVP = GAVGP GVGVP; repeats shown; Ala in first repeat; Val in second repeat; this motif can be repeated 1 or more times
GVGVPGAGVP

SEQ ID NO: 6 - protein, 25 aa, Artificial Sequence
  Synthetic
  MISC_FEATURE (1)..(25): G[V4A1]GVP = GVGVP GVGVP GVGVP GVGVP GAGVP; 5 repeats shown; Val in the first 4 repeats and Ala in the 5th repeat; this motif can be repeated 1 or more times
GVGVPGVGVPGVGVPGVGVPGAGVP

SEQ ID NO: 7 - protein, 25 aa, Artificial Sequence
  Synthetic
AAAAAAAAAAAAAAAAAAAAAAAAA

SEQ ID NO: 8 - protein, 27 aa, Artificial Sequence
  Synthetic
KAAAAAAAAAAAAAAAAAAAAAAAAAK

SEQ ID NO: 9 - protein, 27 aa, Artificial Sequence
  Synthetic
DAAAAAAAAAAAAAAAAAAAAAAAAAK

SEQ ID NO: 10 - protein, 28 aa, Artificial Sequence
  Synthetic
GDAAAAAAAAAAAAAAAAAAAAAAAAAK

SEQ ID NO: 11 - protein, 28 aa, Artificial Sequence
  Synthetic
GKAAAAAAAAAAAAAAAAAAAAAAAAAK

SEQ ID NO: 12 - protein, 622 aa, Artificial Sequence
  Synthetic
MGVGVPGVGVPGVGVPGVGVPGVGVPGVGVPGVGVPGVGVPGVGVPGVGVPGVGVPGVGVPGVGVPGVGVPGVGVPGDAAAAAAAAAAAAAAAAAAAAAAAAAKGVGVPGVGVPGVGVPGVGVPGVGVPGVGVPGVGVPGVGVPGVGVPGVGVPGVGVPGVGVPGVGVPGVGVPGVGVPGDAAAAAAAAAAAAAAAAAAAAAAAAAKGVGVPGVGVPGVGVPGVGVPGVGVPGVGVPGVGVPGVGVPGVGVPGVGVPGVGVPGVGVPGVGVPGVGVPGVGVPGDAAAAAAAAAAAAAAAAAAAAAAAAAKGVGVPGVGVPGVGVPGVGVPGVGVPGVGVPGVGVPGVGVPGVGVPGVGVPGVGVPGVGVPGVGVPGVGVPGVGVPGDAAAAAAAAAAAAAAAAAAAAAAAAAKGVGVPGVGVPGVGVPGVGVPGVGVPGVGVPGVGVPGVGVPGVGVPGVGVPGVGVPGVGVPGVGVPGVGVPGVGVPGDAAAAAAAAAAAAAAAAAAAAAAAAAKGVGVPGVGVPGVGVPGVGVPGVGVPGVGVPGVGVPGVGVPGVGVPGVGVPGVGVPGVGVPGVGVPGVGVPGVGVPGDAAAAAAAAAAAAAAAAAAAAAAAAAKGWP

SEQ ID NO: 13 - protein, 416 aa, Artificial Sequence
  Synthetic
MGVGVPGVGVPGVGVPGVGVPGVGVPGVGVPGVGVPGVGVPGVGVPGVGVPGVGVPGVGVPGVGVPGVGVPGVGVPGDAAAAAAAAAAAAAAAAAAAAAAAAAKGVGVPGVGVPGVGVPGVGVPGVGVPGVGVPGVGVPGVGVPGVGVPGVGVPGVGVPGVGVPGVGVPGVGVPGVGVPGDAAAAAAAAAAAAAAAAAAAAAAAAAKGVGVPGVGVPGVGVPGVGVPGVGVPGVGVPGVGVPGVGVPGVGVPGVGVPGVGVPGVGVPGVGVPGVGVPGVGVPGDAAAAAAAAAAAAAAAAAAAAAAAAAKGVGVPGVGVPGVGVPGVGVPGVGVPGVGVPGVGVPGVGVPGVGVPGVGVPGVGVPGVGVPGVGVPGVGVPGVGVPGDAAAAAAAAAAAAAAAAAAAAAAAAAKGWP

SEQ ID NO: 14 - protein, 416 aa, Artificial Sequence
  Synthetic
MGVGVPGVGVPGVGVPGVGVPGVGVPGVGVPGVGVPGVGVPGVGVPGVGVPGVGVPGVGVPGVGVPGVGVPGVGVPGKAAAAAAAAAAAAAAAAAAAAAAAAAKGVGVPGVGVPGVGVPGVGVPGVGVPGVGVPGVGVPGVGVPGVGVPGVGVPGVGVPGVGVPGVGVPGVGVPGVGVPGKAAAAAAAAAAAAAAAAAAAAAAAAAKGVGVPGVGVPGVGVPGVGVPGVGVPGVGVPGVGVPGVGVPGVGVPGVGVPGVGVPGVGVPGVGVPGVGVPGVGVPGKAAAAAAAAAAAAAAAAAAAAAAAAAKGVGVPGVGVPGVGVPGVGVPGVGVPGVGVPGVGVPGVGVPGVGVPGVGVPGVGVPGVGVPGVGVPGVGVPGVGVPGKAAAAAAAAAAAAAAAAAAAAAAAAAKGWP

SEQ ID NO: 15 - protein, 652 aa, Artificial Sequence
  Synthetic
MGAGVPGVGVPGAGVPGVGVPGAGVPGVGVPGAGVPGVGVPGAGVPGVGVPGAGVPGVGVPGAGVPGVGVPGAGVPGVGVPGDAAAAAAAAAAAAAAAAAAAAAAAAAKGAGVPGVGVPGAGVPGVGVPGAGVPGVGVPGAGVPGVGVPGAGVPGVGVPGAGVPGVGVPGAGVPGVGVPGAGVPGVGVPGDAAAAAAAAAAAAAAAAAAAAAAAAAKGAGVPGVGVPGAGVPGVGVPGAGVPGVGVPGAGVPGVGVPGAGVPGVGVPGAGVPGVGVPGAGVPGVGVPGAGVPGVGVPGDAAAAAAAAAAAAAAAAAAAAAAAAAKGAGVPGVGVPGAGVPGVGVPGAGVPGVGVPGAGVPGVGVPGAGVPGVGVPGAGVPGVGVPGAGVPGVGVPGAGVPGVGVPGDAAAAAAAAAAAAAAAAAAAAAAAAAKGAGVPGVGVPGAGVPGVGVPGAGVPGVGVPGAGVPGVGVPGAGVPGVGVPGAGVPGVGVPGAGVPGVGVPGAGVPGVGVPGDAAAAAAAAAAAAAAAAAAAAAAAAAKGAGVPGVGVPGAGVPGVGVPGAGVPGVGVPGAGVPGVGVPGAGVPGVGVPGAGVPGVGVPGAGVPGVGVPGAGVPGVGVPGDAAAAAAAAAAAAAAAAAAAAAAAAAKGWP

SEQ ID NO: 16 - protein, 436 aa, Artificial Sequence
  Synthetic
MGAGVPGVGVPGAGVPGVGVPGAGVPGVGVPGAGVPGVGVPGAGVPGVGVPGAGVPGVGVPGAGVPGVGVPGAGVPGVGVPGDAAAAAAAAAAAAAAAAAAAAAAAAAKGAGVPGVGVPGAGVPGVGVPGAGVPGVGVPGAGVPGVGVPGAGVPGVGVPGAGVPGVGVPGAGVPGVGVPGAGVPGVGVPGDAAAAAAAAAAAAAAAAAAAAAAAAAKGAGVPGVGVPGAGVPGVGVPGAGVPGVGVPGAGVPGVGVPGAGVPGVGVPGAGVPGVGVPGAGVPGVGVPGAGVPGVGVPGDAAAAAAAAAAAAAAAAAAAAAAAAAKGAGVPGVGVPGAGVPGVGVPGAGVPGVGVPGAGVPGVGVPGAGVPGVGVPGAGVPGVGVPGAGVPGVGVPGAGVPGVGVPGDAAAAAAAAAAAAAAAAAAAAAAAAAKGWP

SEQ ID NO: 17 - protein, 622 aa, Artificial Sequence
  Synthetic
MGVGVPGVGVPGVGVPGVGVPGAGVPGVGVPGVGVPGVGVPGVGVPGAGVPGVGVPGVGVPGVGVPGVGVPGAGVPGDAAAAAAAAAAAAAAAAAAAAAAAAAKGVGVPGVGVPGVGVPGVGVPGAGVPGVGVPGVGVPGVGVPGVGVPGAGVPGVGVPGVGVPGVGVPGVGVPGAGVPGDAAAAAAAAAAAAAAAAAAAAAAAAAKGVGVPGVGVPGVGVPGVGVPGAGVPGVGVPGVGVPGVGVPGVGVPGAGVPGVGVPGVGVPGVGVPGVGVPGAGVPGDAAAAAAAAAAAAAAAAAAAAAAAAAKGVGVPGVGVPGVGVPGVGVPGAGVPGVGVPGVGVPGVGVPGVGVPGAGVPGVGVPGVGVPGVGVPGVGVPGAGVPGDAAAAAAAAAAAAAAAAAAAAAAAAAKGVGVPGVGVPGVGVPGVGVPGAGVPGVGVPGVGVPGVGVPGVGVPGAGVPGVGVPGVGVPGVGVPGVGVPGAGVPGDAAAAAAAAAAAAAAAAAAAAAAAAAKGVGVPGVGVPGVGVPGVGVPGAGVPGVGVPGVGVPGVGVPGVGVPGAGVPGVGVPGVGVPGVGVPGVGVPGAGVPGDAAAAAAAAAAAAAAAAAAAAAAAAAKGWP

SEQ ID NO: 18 - protein, 416 aa, Artificial Sequence
  Synthetic
MGVGVPGVGVPGVGVPGVGVPGAGVPGVGVPGVGVPGVGVPGVGVPGAGVPGVGVPGVGVPGVGVPGVGVPGAGVPGDAAAAAAAAAAAAAAAAAAAAAAAAAKGVGVPGVGVPGVGVPGVGVPGAGVPGVGVPGVGVPGVGVPGVGVPGAGVPGVGVPGVGVPGVGVPGVGVPGAGVPGDAAAAAAAAAAAAAAAAAAAAAAAAAKGVGVPGVGVPGVGVPGVGVPGAGVPGVGVPGVGVPGVGVPGVGVPGAGVPGVGVPGVGVPGVGVPGVGVPGAGVPGDAAAAAAAAAAAAAAAAAAAAAAAAAKGVGVPGVGVPGVGVPGVGVPGAGVPGVGVPGVGVPGVGVPGVGVPGAGVPGVGVPGVGVPGVGVPGVGVPGAGVPGDAAAAAAAAAAAAAAAAAAAAAAAAAKGWP

SEQ ID NO: 19 - protein, 17 aa, Artificial Sequence
  Synthetic
  MISC_FEATURE (2)..(16): Xaa is any amino acid amd any one or all of amino acids 3-16 can be either present or absent
PXXXXXXXXXXXXXXXG

SEQ ID NO: 20 - protein, 47 aa, Artificial Sequence
  Synthetic
  MISC_FEATURE (2)..(15): Asx 2-15 can either be present or absent
  MISC_FEATURE (17)..(31): any amino acid independent of Glx at positions 32-47; and where any one or all amino acids 18-31 can be either present or absent
  MISC_FEATURE (32)..(47): Glx 32-47 are independent of Xaa at positions 17-31; and where any one or all Glx 33-47 can be either present or absent
BBBBBBBBBBBBBBBPXXXXXXXXXXXXXXXGZZZZZZZZZZZZZZZ

SEQ ID NO: 21 - protein, 54 aa, Artificial Sequence
  Synthetic
  MISC_FEATURE (1)..(2): Any or all Asx at positions 1-2 can be either present or absent
  MISC_FEATURE (1)..(54): Motif may repeat 1-50 times
  MISC_FEATURE (4)..(52): Any one or all Ala at positions 4-52 can be either present or absent
  MISC_FEATURE (53)..(54): Any or all Glx at positions 53-54 can be either present or absent
BBAAAAAAAAAAAAAAAAAAAAAAAAAAAAAAAAAAAAAAAAAAAAAAAAAAZZ

SEQ ID NO: 22 - protein, 3 aa, Artificial Sequence
  Synthetic
  MISC_FEATURE (1)..(1): Asx can be either Lys, Arg, Asp, or Glu
  MISC_FEATURE (1)..(2): Motif can repeat from 1-50 times
  MISC_FEATURE (1)..(3): Motif can repeat from 1-50 times
  MISC_FEATURE (2)..(2): Ala can repeat 1-5 times
  MISC_FEATURE (3)..(3): Glx can be Lys, Arg, Asp, or Glu, and can repeat 0-2 times
BAZ